(19) United States
(12) Patent Application Publication
(10) Pub. No.: US 2017/0305819 A1
(43) Pub. Date: Oct. 26, 2017

(54) SELECTIVE PARTIAL HYDROGENATION OF BETA-FARNESENE

(71) Applicant: Novvi LLC, Emeryville, CA (US)

(72) Inventors: Jason WELLS, Fremont, CA (US); Liwenny HO, Oakland, CA (US); Wui Sum Willbe HO, Oakland, CA (US); Shakeel TIRMIZI, Matawan, NJ (US); Eduardo BARALT, Houston, TX (US)

(21) Appl. No.: 15/520,621

(22) PCT Filed: Oct. 20, 2015
(86) PCT No.: PCT/US2015/056430

§ 371 Date: Apr. 20, 2017

Related U.S. Application Data
(60) Provisional application No. 62/066,116, filed on Oct. 20, 2014.

(51) Int. Cl. C07C 11/21; C07C 11/02; C07C 5/05; C07C 11/12

(52) U.S. Cl. CPC C07C 11/21; C07C 11/12; C07C 11/02; C07C 5/05; C07C 2521/04; C07C 2523/44

(57) ABSTRACT

Process for preparing an olefinic product comprising partially hydrogenated β-farnesene in two stages. In the first stage, reaction conditions are controlled to favor the hydrogenation of β-farnesene over auto dimerization and polymerization of β-farnesene. In the second stage, reaction conditions are controlled to favor the hydrogenation of dihydro-β-farnesene and tetrahydro-β-farnesene to form hexahydro-β-hydrofarnesene over the hydrogenation of hexahydro-β-hydrofarnesene to form farnesane.

The present disclosure is generally directed to feedstocks comprising a β-farnesene derivative, methods of making such feedstocks, methods of their use, and compositions comprising such feedstocks. The feedstocks described herein may be used to replace or to supplement olefinic feedstocks derived from fossil fuels.

Olefins are used as raw materials or feedstocks in a variety of industrial processes, such as in the production of fuels, polymers, fatty acids, detergents and working fluids such as lubricants, hydraulic fluids, and compressor oils. Olefinic feedstocks may be derived from a range of sources, including both renewable and non-renewable.

In WO 2012/016172 Platt discloses genetically modified microorganisms that produce increased amounts of acetyl-CoA derived compounds, such as β-farnesene, in industrial fermentation processes from carbon sources.

In WO 2012/141783 Ohler et al. disclose a method for selectively hydrogenating olefinic bond(s) of a conjugated alkene such as β-farnesene to yield mono-olefinic feedstocks. According to Ohler et al., the conjugated alkenes may be the fermentation product of a genetically modified organism. Production by fermentation results in technical grade product containing a variety of natural byproducts. Those byproducts include other hydrocarbons along with alcohols and carboxylic acids. It is also known that conjugated dienes such as β-farnesene will readily undergo a thermal Diels-Alder dimerization to give heavy cyclic products and will undergo auto-oxidation to form hydroperoxides. Many of these byproducts can have detrimental effects on downstream processing and final product quality. Thus, while Ohler et al.'s process offers significant advantages, a need remains for a further improved process for selective partial hydrogenation of β-farnesene.

Among the various aspects of the present disclosure, therefore, may be noted the provision of a process for preparing an olefinic product comprising partially hydrogenated β-farnesene. The process comprises hydrogenating β-farnesene in two stages. In the first stage, reaction conditions are controlled to favor the hydrogenation of β-farnesene over auto dimerization and polymerization of β-farnesene. More specifically, β-farnesene is reacted with hydrogen in the presence of a first stage catalyst to produce a first stage reaction product in which (i) the mass fraction of farnesane relative to the combined amount of β-farnesene, partially hydrogenated β-farnesene and farnesane in the first stage reaction product is no more than 5 wt %, (ii) the mass fraction of β-farnesene relative to the combined amount of β-farnesene, partially hydrogenated β-farnesene and farnesane in the first stage reaction product is no more than 1 wt %, and (iii) the mass fraction of β-farnesene derivatives having a molecular weight greater than farnesane relative to the combined amount of β-farnesene, partially hydrogenated β-farnesene, farnesane and β-farnesene derivatives having a molecular weight greater than farnesane in the first stage reaction product is no more than 2.5 wt %. In the second stage, reaction conditions are controlled to favor the hydrogenation of dihydro-β-farnesene and tetrahydro-β-farnesene to form hexahydro-β-farnesene over the hydrogenation of hexahydro-β-farnesene to form farnesane. More specifically, in the second stage the first stage reaction product is reacted with hydrogen in the presence of a second stage catalyst to produce a second stage reaction product in which (i) the mass fraction of farnesane relative to the combined amount of β-farnesene, partially hydrogenated β-farnesene and farnesane in the second stage reaction product is no more than 7 wt %, (ii) the mass fraction of hexahydro-β-farnesene relative to the combined amount of β-farnesene, partially hydrogenated β-farnesene and farnesane in the second stage reaction product is at least 85 wt %, and (iii) the mass fraction of β-farnesene derivatives having a molecular weight greater than farnesane relative to the combined amount of β-farnesene, partially hydrogenated β-farnesene, farnesane and β-farnesene derivatives having a molecular weight greater than farnesane in the second stage reaction product is no more than 2.5 wt %.

In one embodiment, the β-farnesene is produced by one or more microorganisms. For example, the β-farnesene can be produced by a bioengineered microorganism, i.e., a microorganism engineered to produce the conjugated diene starting material, or a precursor thereof. In particular embodiments, the microorganism produces the β-farnesene starting material from a renewable carbon source. In such embodiments, the present methods provide renewable sources for the resulting olefinic products.

Another aspect of the present disclosure is an olefinic product comprising partially hydrogenated β-farnesene. In the olefinic product, (i) the mass fraction of farnesane relative to the combined amount of β-farnesene, partially hydrogenated β-farnesene and farnesane is no more than 1 wt %, (ii) the mass fraction of β-farnesene relative to the combined amount of β-farnesene, partially hydrogenated β-farnesene and farnesane is no more than 5 wt %, and (iii) the mass fraction of β-farnesene derivatives having a molecular weight greater than farnesane relative to the combined amount of β-farnesene, partially hydrogenated β-farnesene, farnesane and β-farnesene derivatives having a molecular weight greater than farnesane is no more than 2.5 wt %.

Another aspect of the present disclosure is an olefinic product comprising hexahydro-β-farnesene. In the olefinic product, (i) the mass fraction of farnesane relative to the combined amount of β-farnesene, partially hydrogenated β-farnesene and farnesane in the second stage reaction product is no more than 7 wt %, (ii) the mass fraction of hexahydro-β-farnesene to the combined amount of β-farnesene, partially hydrogenated β-farnesene and farnesane in the second stage reaction product is at least 85 wt %, and (iii) the mass fraction of β-farnesene derivatives having a molecular weight greater than farnesane relative to the combined amount of β-farnesene, partially hydrogenated β-farnesene, farnesane and β-farnesene derivatives having a molecular weight greater than farnesane in the second stage reaction product is no more than 2.5 wt %.

In certain embodiments, the olefinic products have little or no sulfur or aromatic content. In certain embodiments, the olefinic products have little or no sulfur content. In certain embodiments, the olefinic products have little or no aromatic content. In certain embodiments, the olefinic products have little or no sulfur content and little or no aromatic content. Exemplary amounts of sulfur and/or aromatic content are described elsewhere herein.

### BRIEF DESCRIPTION OF THE DRAWINGS

FIG. 1 is a schematic process flow diagram for a two-stage process for the partial hydrogenation of β-farnesene in accordance with one aspect of the present disclosure.

FIG. 2 is a non-limiting example of a process flow diagram for partially hydrogenating β-farnesene in accordance with one embodiment of the present disclosure.

FIG. 3 is a non-limiting example of a process flow diagram for partially hydrogenating β-farnesene in accordance with an alternative embodiment of the present disclosure.

FIG. 4 is a non-limiting example of a process flow diagram for partially hydrogenating β-farnesene in accordance with an alternative embodiment of the present disclosure.

FIG. 5 is a non-limiting example of a process flow diagram for partially hydrogenating β-farnesene in accordance with an alternative embodiment of the present disclosure.

FIG. 6 is a non-limiting example of a process flow diagram for partially hydrogenating β-farnesene in accordance with an alternative embodiment of the present disclosure.

FIG. 7 is a non-limiting example of a process flow diagram for partially hydrogenating β-farnesene in accordance with an alternative embodiment of the present disclosure.

FIG. 8 is a non-limiting example of a process flow diagram for partially hydrogenating β-farnesene in accordance with an alternative embodiment of the present disclosure.

Corresponding reference characters indicate corresponding parts throughout the drawings.

### Definitions

The following definitions and methods are provided to better define the present invention and to guide those of ordinary skill in the art in the practice of the present invention. Unless otherwise noted, terms are to be understood according to conventional usage by those of ordinary skill in the relevant art.

β-farnesene refers to a compound having the molecular formula C₁₅H₂₄ and the structural formula:

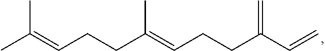

and includes the stereoisomers thereof.

“Dimer” or “Thermal Dimer” as used herein refers to a dimer derivative of β-farnesene.

“Dihydro-β-farnesene” is a hydrogenated derivative of β-farnesene having the molecular formula C₁₅H₂₆, and includes each of the stereoisomers thereof.

“Farnesane” refers to a compound having the molecular formula

C₁₅H₃₂ and the structural formula:

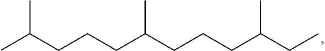

and includes the stereoisomers thereof.

“Hexahydro-β-farnesene” is a hydrogenated derivative of β-farnesene having the molecular formula C₁₅H₃₀, and includes each of the stereoisomers thereof.

“Hydrogenated β-farnesene” refers to a hydrogenated derivative of β-farnesene wherein at least one the four carbon-carbon double bonds of β-farnesene is hydrogenated to form a saturated (sp³ hybridized) carbon-carbon bond. Hydrogenated β-farnesene encompasses dihydro-β-farnesene, tetrahydro-β-farnesene, hexahydro-β-farnesene and farnesane, and each of the combinations thereof.

“Partially hydrogenated β-farnesene” refers to a hydrogenated β-farnesene derivative in which one, two, or three of the four carbon-carbon double bonds of β-farnesene is hydrogenated to form a saturated (sp³ hybridized) carbon-carbon bond. Partially hydrogenated β-farnesene derivatives include dihydro-β-farnesene, tetrahydro-β-farnesene, and hexahydro-β-farnesene but not farnesane.

“Polymer” refers to a polymer derivative of β-farnesene.

“Recycling fraction” refers to a fraction of a product composition that is separated from an effluent of a hydrogenation reaction provided herein and recycled as a diluent in the hydrogenation reaction.

“Tetrahydro-β-farnesene” is a hydrogenated β-farnesene derivative having the molecular formula C₁₅H₂₈, and includes each of the stereoisomers thereof.

In the present disclosure, whenever a numerical range with a lower limit R^{L} and an upper limit R^{U} is disclosed, any number falling within the range is specifically disclosed. In particular, the following numbers Rₖ within the range are specifically disclosed: Rₖ=R^{L}+k·(R^{U}−R^{L}) wherein k is a variable ranging from 1% to 100% with a 1% increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Further, any numerical range defined by any two numbers Rₖ as defined above is also specifically disclosed herein.

A composition that consists “predominantly” of a component refers to a composition comprising 60% or more of that component, unless indicated otherwise.

As used herein, “%” refers to % measured as wt. % or as area % by GC-FID or GPC, unless specifically indicated otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and not exclusive (i.e., there may be other elements in addition to the recited elements). Additionally, the use of the singular includes the plural and plural encompasses singular, unless specifically stated otherwise. Furthermore, the use of “or” means “and/or” unless specifically stated otherwise.

### DETAILED DESCRIPTION

At elevated temperatures (e.g., temperatures of at least about 60° C. and as a function of increasing temperature and concentration, β-farnesene participates in dimerization, cyclization, isomerization, or other competing or degradation processes to form undesired reaction products such as dimers and other higher molecular weight oligomers/polymers. Advantageously, the β-farnesene conjugated carbon-carbon double bond tends to be more reactive with hydrogen than the other β-farnesene carbon-carbon double bonds. In general, therefore, the formation of dimers and other β-farnesene derivatives having a molecular weight greater than farnesane can be minimized by controlling reaction conditions selective for the hydrogenation of the β-farnesene conjugated carbon-carbon double bond over the formation of dimers and other β-farnesene derivatives having a molecular weight greater than farnesane until the reaction mixture contains a sufficiently low concentration of species containing a conjugated carbon-carbon double bond (i.e., β-farnesene). Once the reaction mixture contains a sufficiently low concentration of β-farnesene conjugated carbon-carbon double bonds, reaction conditions may be adjusted to increase the rate of hydrogenation of dihydro-β-farnesene and tetrahydro-β-farnesene to form hexahydro-β-farnesene without concern for the formation of β-farnesene derivatives having a molecular weight greater than farnesane.

In accordance with one aspect of the present disclosure, therefore, a hydrogenated β-farnesene product having a relatively high concentration of hexahydro-β-farnesene (relative to other partially hydrogenated β-farnesene derivatives and/or farnesane) and a relatively low concentration of β-farnesene derivatives having a molecular weight greater than farnesane (relative to partially hydrogenated β-farnesene derivatives and/or farnesane) may be prepared from a β-farnesene starting material. These relative concentrations may be achieved by hydrogenating the β-farnesene starting material in two stages.

In the first stage a β-farnesene feedstock is reacted with hydrogen in the presence of a first stage catalyst to produce a first stage reaction product having a large mass fraction of partially hydrogenated β-farnesene relative to the amount of (β-farnesene, farnesane and β-farnesene derivatives having a molecular weight greater than farnesane in first stage reaction product. For example, in one embodiment a β-farnesene feedstock is reacted in the first stage with at least about 0.9 equivalents of hydrogen per equivalent of β-farnesene in the feedstock. By way of further example, in one such embodiment the β-farnesene feedstock is reacted with at least about 1 equivalents of hydrogen per equivalent of β-farnesene in the feedstock in the first stage. By way of further example, in one such embodiment the β-farnesene feedstock is reacted with at least about 1.1 equivalents of hydrogen per equivalent of β-farnesene in the feedstock in the first stage. By way of further example, in one such embodiment the β-farnesene feedstock is reacted with at least about 1.2 equivalents of hydrogen per equivalent of β-farnesene in the feedstock in the first stage. Typically, however, the β-farnesene feedstock is reacted with less than 2 equivalents of hydrogen per equivalent of β-farnesene in the feedstock in the first stage. For example, in some embodiments the β-farnesene feedstock is reacted with less than 1.75 equivalents of hydrogen per equivalent of β-farnesene in the feedstock in the first stage. By way of further example, in some embodiments the β-farnesene feedstock is reacted with less than 1.6 equivalents of hydrogen per equivalent of β-farnesene in the feedstock in the first stage. By way of further example, in some embodiments the β-farnesene feedstock is reacted with less than 1.5 equivalents of hydrogen per equivalent of β-farnesene in the feedstock in the first stage. By way of further example, in some embodiments the β-farnesene feedstock is reacted with less than 1.4 equivalents of hydrogen per equivalent of β-farnesene in the feedstock in the first stage. By way of further example, in some embodiments the β-farnesene feedstock is reacted with less than 1.3 equivalents of hydrogen per equivalent of β-farnesene in the feedstock in the first stage. By way of further example, in some embodiments the β-farnesene feedstock is reacted with less than 1.25 equivalents of hydrogen per equivalent of β-farnesene in the feedstock in the first stage.

In one embodiment the mass fraction of partially hydrogenated β-farnesene (relative to the combined amount of β-farnesene, partially hydrogenated β-farnesene, and farnesane) in the first stage reaction product is at least 95%. By way of further example, in one such embodiment the mass fraction of β-farnesene (relative to the combined amount of β-farnesene, partially hydrogenated β-farnesene, and farnesane) in the first stage reaction product is no more than 5 wt %. By way of further example, in one such embodiment the mass fraction of farnesane (relative to the combined amount of β-farnesene, partially hydrogenated β-farnesene, and farnesane) in the first stage reaction product is no more than 1 wt %. By way of further example, in one such embodiment the mass fraction of β-farnesene derivatives having a molecular weight greater than farnesane (relative to the combined amount of β-farnesene, partially hydrogenated β-farnesene, farnesane and β-farnesene derivatives having a molecular weight greater than farnesane) in the first stage reaction product is no more than 2.5 wt %.

In certain embodiments, the mass fraction of partially hydrogenated β-farnesene (relative to the combined amount of β-farnesene, partially hydrogenated β-farnesene, and farnesane) in the first stage reaction product is greater than 97 wt %. For example, the mass fraction of partially hydrogenated β-farnesene (relative to the combined amount of β-farnesene, partially hydrogenated β-farnesene, and farnesane) in the first stage reaction product is greater than 98 wt %. By way of further example in certain embodiments the mass fraction of partially hydrogenated β-farnesene (relative to the combined amount of β-farnesene, partially hydrogenated β-farnesene, and farnesane) in the first stage reaction product is greater than 99 wt %. By way of further example in certain embodiments the mass fraction of partially hydrogenated β-farnesene (relative to the combined amount of β-farnesene, partially hydrogenated β-farnesene, and farnesane) in the first stage reaction product is greater than 99.5 wt %. By way of further example in certain embodiments the mass fraction of partially hydrogenated β-farnesene (relative to the combined amount of β-farnesene, partially hydrogenated β-farnesene, and farnesane) in the first stage reaction product is greater than 99.9 wt %.

In certain embodiments, the mass fraction of β-farnesene (relative to the combined amount of β-farnesene, partially hydrogenated β-farnesene, and farnesane) in the first stage reaction product is no more than 4 wt %. For example, in certain embodiments the mass fraction of β-farnesene (relative to the combined amount of β-farnesene, partially hydrogenated β-farnesene, and farnesane) in the first stage reaction product is no more than 3 wt %. By way of further example, in certain embodiments the mass fraction of β-farnesene (relative to the combined amount of β-farnesene, partially hydrogenated β-farnesene, and farnesane) in the first stage reaction product is no more than 2 wt %. By way of further example, in certain embodiments the mass fraction of β-farnesene (relative to the combined amount of β-farnesene, partially hydrogenated β-farnesene, and farnesane) in the first stage reaction product is no more than 1 wt %. By way of further example, in certain embodiments the mass fraction of β-farnesene (relative to the combined amount of β-farnesene, partially hydrogenated β-farnesene, and farnesane) in the first stage reaction product is no more than 0.5 wt %. By way of further example, in certain embodiments the mass fraction of β-farnesene (relative to the combined amount of β-farnesene, partially hydrogenated β-farnesene, and farnesane) in the first stage reaction product is no more than 0.25 wt %. By way of further example, in certain embodiments the mass fraction of β-farnesene (relative to the combined amount of β-farnesene, partially hydrogenated β-farnesene, and farnesane) in the first stage reaction product is no more than 0.1 wt %. By way of further example, in certain embodiments β-farnesene is not detectible in the first stage reaction product.

In certain embodiments, the mass fraction of farnesane (relative to the combined amount of β-farnesene, partially hydrogenated β-farnesene, and farnesane) in the first stage reaction product is no more than 0.75 wt %. For example, in certain embodiments the mass fraction of farnesane (relative to the combined amount of β-farnesene, partially hydrogenated β-farnesene, and farnesane) in the first stage reaction product is no more than 0.5 wt %. By way of further example, in certain embodiments the mass fraction of farnesane (relative to the combined amount of β-farnesene, partially hydrogenated β-farnesene, and farnesane) in the first stage reaction product is no more than 0.25 wt %. By way of further example, in certain embodiments the mass fraction of farnesane (relative to the combined amount of β-farnesene, partially hydrogenated β-farnesene, and farnesane) in the first stage reaction product is no more than 0.1 wt %. By way of further example, in certain embodiments the mass fraction of farnesane (relative to the combined amount of β-farnesene, partially hydrogenated β-farnesene, and farnesane) in the first stage reaction product is no more than 0.05 wt %. By way of further example, in certain embodiments the mass fraction of farnesane (relative to the combined amount of β-farnesene, partially hydrogenated β-farnesene, and farnesane) in the first stage reaction product is not detectible.

In certain embodiments, the mass fraction of β-farnesene derivatives having a molecular weight greater than farnesane (relative to the combined amount of β-farnesene, partially hydrogenated β-farnesene, farnesane and β-farnesene derivatives having a molecular weight greater than farnesane) in the first stage reaction product is no more than 2.5 wt %. For example, in certain embodiments the mass fraction of farnesane (relative to the combined amount of β-farnesene, partially hydrogenated β-farnesene, and farnesane) in the first stage reaction product is no more than 2 wt %. By way of further example, in certain embodiments the mass fraction of farnesane (relative to the combined amount of β-farnesene, partially hydrogenated β-farnesene, and farnesane) in the first stage reaction product is no more than 1.5 wt %. By way of further example, in certain embodiments the mass fraction of farnesane (relative to the combined amount of β-farnesene, partially hydrogenated β-farnesene, and farnesane) in the first stage reaction product is no more than 1 wt %. By way of further example, in certain embodiments the mass fraction of farnesane (relative to the combined amount of β-farnesene, partially hydrogenated β-farnesene, and farnesane) in the first stage reaction product is no more than 0.5 wt %. By way of further example, in certain embodiments the mass fraction of farnesane (relative to the combined amount of β-farnesene, partially hydrogenated β-farnesene, and farnesane) in the first stage reaction product is no more than 0.25 wt %. By way of further example, in certain embodiments the mass fraction of farnesane (relative to the combined amount of β-farnesene, partially hydrogenated β-farnesene, and farnesane) in the first stage reaction product is no more than 0.1 wt %. By way of further example, in certain embodiments the mass fraction of farnesane (relative to the combined amount of β-farnesene, partially hydrogenated β-farnesene, and farnesane) in the first stage reaction product is not detectible.

In certain embodiments, the first stage produces a reaction product comprising a high concentration of partially hydrogenated β-farnesene that predominantly comprises dihydro-β-farnesene. Thus, for example, in one embodiment the first stage reaction product contains at least 85 wt % dihydro-β-farnesene (relative to the combined amount of β-farnesene, partially hydrogenated β-farnesene, and farnesane). By way of further example in one such embodiment the first stage reaction product contains at least 90 wt % dihydro-β-farnesene (relative to the combined amount of β-farnesene, partially hydrogenated β-farnesene, and farnesane). By way of further example in one such embodiment the first stage reaction product contains at least 92 wt % dihydro-β-farnesene (relative to the combined amount of β-farnesene, partially hydrogenated β-farnesene, and farnesane). By way of further example in one such embodiment the first stage reaction product contains at least 94 wt % dihydro-β-farnesene (relative to the combined amount of β-farnesene, partially hydrogenated β-farnesene, and farnesane). By way of further example in one such embodiment the first stage reaction product contains at least 96 wt % dihydro-β-farnesene (relative to the combined amount of β-farnesene, partially hydrogenated β-farnesene, and farnesane).

In certain presently preferred embodiments the first stage reaction product is predominantly partially hydrogenated β-farnesene and comprises small amounts of β-farnesene and β-farnesene derivatives having a molecular weight greater than farnesane. For example, the first stage reaction product may comprise a reaction product corresponding to First Stage Exemplary Product A, B or C in Table I wherein (i) the extent of hydrogenation is the ratio of the number of equivalents of hydrogen and β-farnesene (H₂:β-farnesene) reacted to obtain the exemplary reaction product, (ii) the weight percentage of partially hydrogenated β-farnesene, β-farnesene and farnesane relative to the combined amount of β-farnesene, partially hydrogenated β-farnesene, and farnesane in the exemplary reaction product and (iii) the weight percentage of β-farnesene derivatives having a molecular weight greater than farnesane is relative to the combined amount of β-farnesene, partially hydrogenated β-farnesene, farnesane and β-farnesene derivatives having a molecular weight greater than farnesane in the exemplary reaction product.

TABLE I
Exemplary Stage One Reaction Product Compositions

| Exemplary | | | | wt % β-farnesene |
|---|---|---|---|---|
| First | | | | derivatives |
| Stage | | | | having a molecular |
| Reaction | Extent of | wt % of | wt % of | weight greater |
| Product | Hydrogenation | Farnesene | Farnesane | than farnesane |
| A | 0.9-2.0 H | 0-10% | ≦1% | 0-2.5% |
| B | 1-1.5 H | ≦1% | ≦0.1% | 0-1% |
| C | 1.0-1.1 H | not | not | ≦0.05% |
| | | detectable | detectable | |

Upon completion of the first stage, the first stage reaction product is reacted with hydrogen in the presence of a second stage catalyst in the second stage to produce a second stage reaction product having a large mass fraction of hexahydro-β-farnesene relative to the amount of other partially hydrogenated β-farnesene species, farnesane, and β-farnesene derivatives having a molecular weight greater than farnesane in second stage reaction product. As described above, in the first stage the β-farnesene is reacted with up to about 2 equivalents of hydrogen per equivalent of (β-farnesene in the feedstock (typically in the range of about 1.1 to about 1.2 equivalents of hydrogen per equivalent of β-farnesene in the feedstock). In the second stage, the first stage reaction product is reacted with sufficient hydrogen such that cumulatively, in the two stages, the β-farnesene is reacted with up to about 4 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with up to about 3.5 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with up to about 3.4 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with up to about 3.3 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with up to about 3.2 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with up to about 3.1 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with up to about 3.0 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with up to about 2.9 equivalents of hydrogen per equivalent of β-farnesene in the feedstock. By way of further example, in one such embodiment the first stage reaction product is reacted with sufficient hydrogen in the second stage, such that, cumulatively, in the two stages, the β-farnesene is reacted with 2.95 to about 3.05 equivalents of hydrogen per equivalent of β-farnesene in the feedstock.

In one embodiment, the mass fraction of hexahydro-β-farnesene (relative to the combined amount of β-farnesene, partially hydrogenated β-farnesene, and farnesane) in the second stage reaction product is at least 85 wt %. By way of further example, in one such embodiment the mass fraction of farnesane (relative to the combined amount of β-farnesene, partially hydrogenated β-farnesene, and farnesane) in the second stage reaction product is no more than 7 wt %. By way of further example, in one such embodiment the mass fraction of β-farnesene derivatives having a molecular weight greater than farnesane (relative to the combined amount of β-farnesene, partially hydrogenated β-farnesene, farnesane and β-farnesene derivatives having a molecular weight greater than farnesane) in the second stage reaction product) is no more than 2.5 wt %.

In certain embodiments, the second stage reaction product contains more than 85 wt % hexahydro-β-farnesene (relative to the combined amount of β-farnesene, partially hydrogenated β-farnesene, and farnesane) in the second stage reaction product. For example, in one such embodiment the second stage reaction product contains at least 87 wt % hexahydro-β-farnesene (relative to the combined amount of β-farnesene, partially hydrogenated β-farnesene, and farnesane) in the second stage reaction product. By way of further example in one such embodiment the second stage reaction product contains at least 88 wt % hexahydro-β-farnesene (relative to the combined amount of β-farnesene, partially hydrogenated β-farnesene, and farnesane) in the second stage reaction product. By way of further example in one such embodiment the second stage reaction product contains at least 89 wt % hexahydro-β-farnesene (relative to the combined amount of β-farnesene, partially hydrogenated β-farnesene, and farnesane) in the second stage reaction product. By way of further example in one such embodiment the second stage reaction product contains at least 90 wt % hexahydro-β-farnesene (relative to the combined amount of β-farnesene, partially hydrogenated β-farnesene, and farnesane) in the second stage reaction product. By way of further example in one such embodiment the second stage reaction product contains at least 91 wt % hexahydro-β-farnesene (relative to the combined amount of β-farnesene, partially hydrogenated β-farnesene, and farnesane) in the second stage reaction product. By way of further example in one such embodiment the second stage reaction product contains at least 92 wt % hexahydro-β-farnesene (relative to the combined amount of β-farnesene, partially hydrogenated β-farnesene, and farnesane) in the second stage reaction product. By way of further example in one such embodiment the second stage reaction product contains at least 93 wt % hexahydro-β-farnesene (relative to the combined amount of β-farnesene, partially hydrogenated β-farnesene, and farnesane) in the second stage reaction product. By way of further example in one such embodiment the second stage reaction product contains at least 94 wt % hexahydro-β-farnesene (relative to the combined amount of β-farnesene, partially hydrogenated β-farnesene, and farnesane) in the second stage reaction product. By way of further example in one such embodiment the second stage reaction product contains at least 95 wt % hexahydro-β-farnesene (relative to the combined amount of β-farnesene, partially hydrogenated β-farnesene, and farnesane) in the second stage reaction product.

In certain embodiments the second stage reaction product contains less than 7 wt % farnesane (relative to the combined amount of β-farnesene, partially hydrogenated β-farnesene, and farnesane) in the second stage reaction product. By way of further example in one such embodiment the second stage reaction product contains less than 6 wt % farnesane (relative to the combined amount of β-farnesene, partially hydrogenated β-farnesene, and farnesane) in the second stage reaction product. By way of further example in one such embodiment the second stage reaction product contains less than 5 wt % farnesane (relative to the combined amount of β-farnesene, partially hydrogenated β-farnesene, and farnesane) in the second stage reaction product. By way of further example in one such embodiment the second stage reaction product contains less than 4 wt % farnesane (relative to the combined amount of β-farnesene, partially hydrogenated β-farnesene, and farnesane) in the second stage reaction product. By way of further example in one such embodiment the second stage reaction product contains less than 3 wt % farnesane (relative to the combined amount of β-farnesene, partially hydrogenated β-farnesene, and farnesane) in the second stage reaction product. By way of further example in one such embodiment the second stage reaction product contains less than 2 wt % farnesane (relative to the combined amount of β-farnesene, partially hydrogenated β-farnesene, and farnesane) in the second stage reaction product. By way of further example in one such embodiment the second stage reaction product contains less than 1 wt % farnesane (relative to the combined amount of β-farnesene, partially hydrogenated β-farnesene, and farnesane) in the second stage reaction product.

In one embodiment the second stage reaction product contains less than 2.5 wt % β-farnesene derivatives having a molecular weight greater than farnesane (relative to the combined amount of β-farnesene, partially hydrogenated β-farnesene, farnesane and β-farnesene derivatives having a molecular weight greater than farnesane in the second stage reaction product). By way of further example in one such embodiment the second stage reaction product contains less than 1.5 wt % β-farnesene derivatives having a molecular weight greater than farnesane (relative to the combined amount of β-farnesene, partially hydrogenated β-farnesene, farnesane and β-farnesene derivatives having a molecular weight greater than farnesane in the second stage reaction product). By way of further example in one such embodiment the first stage reaction product contains less than 1 wt % β-farnesene derivatives having a molecular weight greater than farnesane (relative to the combined amount of β-farnesene, partially hydrogenated β-farnesene, farnesane and β-farnesene derivatives having a molecular weight greater than farnesane in the second stage reaction product). By way of further example in one such embodiment the first stage reaction product contains less than 0.5 wt % β-farnesene derivatives having a molecular weight greater than farnesane (relative to the combined amount of β-farnesene, partially hydrogenated β-farnesene, farnesane and β-farnesene derivatives having a molecular weight greater than farnesane in the second stage reaction product). By way of further example in one such embodiment the first stage reaction product contains less than 0.1 wt % β-farnesene derivatives having a molecular weight greater than farnesane (relative to the combined amount of β-farnesene, partially hydrogenated β-farnesene, farnesane and β-farnesene derivatives having a molecular weight greater than farnesane in the second stage reaction product). By way of further example in one such embodiment the first stage reaction product contains less than 0.05 wt % β-farnesene derivatives having a molecular weight greater than farnesane (relative to the combined amount of β-farnesene, partially hydrogenated β-farnesene, farnesane and β-farnesene derivatives having a molecular weight greater than farnesane in the second stage reaction product). By way of further example in one such embodiment the first stage reaction product contains no detectible β-farnesene derivatives having a molecular weight greater than farnesane (relative to the combined amount of β-farnesene, partially hydrogenated (β-farnesene, farnesane and β-farnesene derivatives having a molecular weight greater than farnesane in the second stage reaction product).

In one embodiment the second stage reaction product contains less than 0.05 wt % β-farnesene (relative to the combined amount of β-farnesene, partially hydrogenated β-farnesene, and farnesane). By way of further example in one such embodiment the second stage reaction product contains no detectible β-farnesene.

In certain presently preferred embodiments the second stage reaction product is predominantly hexahydro-β-farnesene and comprises small amounts of farnesane and β-farnesene derivatives having a molecular weight greater than farnesane. For example, the first stage reaction product may comprise a reaction product corresponding to Second Stage Exemplary Product D, E or F in Table II wherein (i) the extent of hydrogenation is the ratio of the total number of equivalents of hydrogen and β-farnesene (H₂:β-farnesene) reacted to obtain the exemplary reaction product in stages 1 and 2, (ii) the weight percentage of hexahydro-β-farnesene, tetrahydro-β-farnesene and farnesane relative to the combined amount of partially hydrogenated β-farnesene and farnesane in the exemplary reaction product and (iii) the weight percentage of β-farnesene derivatives having a molecular weight greater than farnesane (i.e., polymer and dimer) relative to the combined amount of partially hydrogenated β-farnesene, farnesane and β-farnesene derivatives having a molecular weight greater than farnesane in the exemplary reaction product.

TABLE II
Exemplary Stage Two Reaction Product Compositions

| Exemplary | | | | | | |
|---|---|---|---|---|---|---|
| Second | | | | | | |
| Stage | | wt % of | wt % of | | | |
| Reaction | Extent of | Hexahydro- | Tetrahydro- | wt % of | wt % | wt % |
| Product | Hydrogenation | farnesene | farnesene | Farnesane | polymer | Dimer |
| D | 2.5-3.4 H | >85% | <10% | <5% | 0-0.5% | 0-2% |
| E | 2.7-3.0 H | ≧85% | ≦8% | <3% | 0-0.5% | 0-2% |
| F | 2.95-3.06 H | ≧90% | ≦6% | <4% | 0.01%** | 0-.05% |

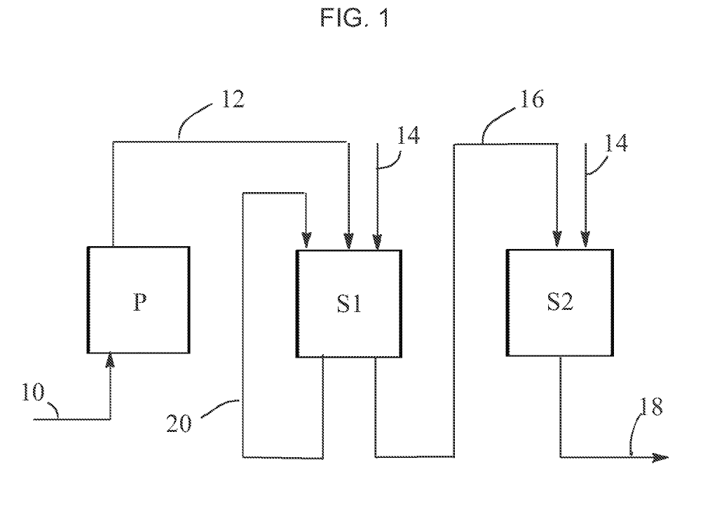

FIG. 1 schematically illustrates one exemplary embodiment for the hydrogenation of β-farnesene in stages in accordance with one embodiment of the present disclosure. Feed stream 10 containing β-farnesene starting material is pretreated in step P to remove impurities such as acids, alcohols and/or epoxides in the β-farnesene that can poison or deactivate the hydrogenation catalyst(s) in downstream operations. Pretreated β-farnesene feed 12, hydrogen 14 and hydrogenation catalyst (not shown) are combined to selectively hydrogenate the β-farnesene in a first hydrogenation stage S1 to produce a first stage reaction product 16 as previously described. First stage reaction product 16, hydrogen 14 and hydrogenation catalyst (not shown) are combined in second hydrogenation stage S2 to selectively hydrogenate dihydro-β-farnesene and tetrahydro-β-farnesene (comprised by the first stage reaction product) to produce second stage reaction product 18 as previously described.

In some embodiments, the β-farnesene starting material comprised by feed stream 10 is a substantially pure stereoisomer of β-farnesene. Substantially pure β-farnesene refers to compositions comprising at least 80%, at least 90%, at least 95%, at least 97%, at least 98% or at least 99% β-farnesene by weight, based on total weight of the farnesene. In other embodiments, the β-farnesene starting material comprised by feed stream 10 is a mixture of stereoisomers, such as s-cis and s-trans isomers. In some embodiments, the amount of each of the stereoisomers in such a mixture is independently from about 0.1 wt. % to about 99.9 wt. %, from about 0.5 wt. % to about 99.5 wt. %, from about 1 wt. % to about 99 wt. %, from about 5 wt. % to about 95 wt. %, from about 10 wt. % to about 90 wt. %, or from about 20 wt. % to about 80 wt. %, based on the total weight of the mixture of β-farnesene stereoisomers.

The β-farnesene starting material may be obtained from any suitable source. In some embodiments, the β-farnesene starting material is obtained from naturally occurring plants or marine species. For example, β-farnesene can be obtained or derived from sugar fermentation or naturally-occurring oils.

In some embodiments, the β-farnesene starting material is obtained using genetically modified organisms that are grown using renewable carbon sources (e.g., sugar cane). In certain embodiments, β-farnesene is prepared by contacting a cell capable of making β-farnesene with a suitable carbon source under conditions suitable for making β-farnesene. Non-limiting examples of β-farnesene obtained using genetically modified organisms are provided in U.S. Pat. No. 7,399,323, U.S. Pat. Publ. Nos. 2008/0274523 and 2009/0137014, and International Patent Publication WO 2007/140339, and International Patent Publication WO 2007/139924, each of which is incorporated herein by reference in its entirety. Any carbon source that can be converted into β-farnesene can be used herein. In some embodiments, the carbon source is a fermentable carbon source (e.g., sugars), a non-fermentable carbon source or a combination thereof. A non-fermentable carbon source is a carbon source that cannot be converted by an organism into ethanol. Non-limiting examples of suitable non-fermentable carbon sources include acetate, glycerol, lactate and ethanol.

Advantageously, in any of the embodiments described herein, the β-farnesene starting material may be produced using renewable resources. As used herein, a “renewable carbon” source refers to a carbon source that is made from modern carbon that can be regenerated within several months, years or decades rather than a carbon source derived from fossil fuels (e.g., petroleum) that takes typically a million years or more to regenerate. The terms “renewable carbon” and “biobased carbon” are used interchangeably herein. “Atmospheric carbon” refers to carbon atoms from carbon dioxide molecules that have been free in earth's atmosphere recently, e.g., in the most recent few decades. For example, β-farnesene used in any one of the embodiments described herein can be made from microorganisms, including bioengineered microorganisms, using a renewable carbon source.

Renewable carbon content can be measured using any suitable method. For example, renewable carbon content can be measured according to ASTM D6866-11, “Standard Test Methods for Determining the Biobased Content of Solid, Liquid, and Gaseous Samples Using Radiocarbon Analysis,” published by ASTM International, which is incorporated herein by reference in its entirety. Some carbon in atmospheric carbon dioxide is the radioactive ¹⁴C isotope, having a half-life of about 5730 years. Atmospheric carbon dioxide is utilized by plants to make organic molecules. The atmospheric ¹⁴C becomes part of biologically produced substances. As the biologically produced organic molecules degrade to produce carbon dioxide into the atmosphere, no net increase of carbon in the atmosphere is produced as a result, which may control or diminish undesired climate effects that may result when molecules produced from fossil fuels degrade to produce carbon dioxide to increase carbon in the atmosphere.

In one embodiment, the β-farnesene starting material comprises virtually no sulfur and no aromatic compounds, making them environmentally preferable over conventional olefins derived from fossil fuels, which in many cases contain sulfur and aromatics, such as naphthalenes. In certain embodiments, the β-farnesene starting material comprises less than about 10 ppm sulfur, less than about 1 ppm sulfur, less than about 100 ppb sulfur, less than about 10 ppb sulfur or less than about 1 ppb sulfur. In certain embodiments, the β-farnesene starting material comprises less than about 10 ppm aromatics, less than about 1 ppm aromatics, less than about 100 ppb aromatics, less than about 10 ppb aromatics or less than about 1 ppb aromatics. In certain embodiments, the β-farnesene starting material comprise less than about 10 ppm sulfur and less than about 10 ppm aromatics, less than about 1 ppm sulfur and less than about 1 ppm aromatics, less than about 100 ppb sulfur and less than about 100 ppb aromatics, less than about 10 ppb sulfur and less than about 10 ppb aromatics, or less than about 1 ppb sulfur and less than about 1 ppb aromatics.

Referring again to FIG. 1, in one embodiment, feed stream 10 is treated in an adsorption purification column (step P). In one presently preferred embodiment, the resulting pre-treated β-farnesene feed 12 satisfies the following specifications:

Purity:≧97% wt β-farnesene

80-200 ppm 4-tert-butylcatechol (“TBC”)

Peroxides<4 ppm,

Water<400 ppm (and no free water)

Total Acid Number (“TAN”)≦0.1

Thermal dimer <1%, Polymer <0.5%

Non-detectible sulphur, nitrogen and phosphorus-containing compounds.

Non-detectible cations, e.g., Na, K, Ca, Mg.

In the first hydrogenation stage S1, pre-treated β-farnesene feed 12 is reacted with hydrogen 14 in the presence of a first stage catalyst (not shown) under conditions selective for the hydrogenation of β-farnesene over the formation of β-farnesene derivatives having a molecular weight greater than farnesane. In general, selectivity for the hydrogenation of β-farnesene over the formation of β-farnesene derivatives having a molecular weight greater than farnesane in the first stage is influenced by several parameters. For example, selectivity for the hydrogenation of β-farnesene over the formation of β-farnesene derivatives having a molecular weight greater than farnesane increases as a function of decreasing temperature, decreasing β-farnesene concentration, increased mixing of the reaction mixture, and increasing catalyst concentration in the reaction mixture. Each of these parameters may be controlled independently, and/or in concert in the first stage of the hydrogenation process, to control selectivity.

In general, the temperature of the first stage reaction mixture (containing β-farnesene, hydrogen, and first stage hydrogenation catalyst) may be gradually increased from a temperature at or near room temperature to a maximum temperature of about 120° C. as the amount of β-farnesene decreases and the amount of partially hydrogenated β-farnesene increases in the first stage reaction mixture. In one embodiment, the temperature of the reaction mixture during the first stage is allowed to increase as the extent of hydrogenation increases. For example, in one embodiment, the temperature of the first stage reaction mixture is gradually increased from room temperature (25° C.) to a temperature in the range of 80 to 100° C. as hydrogenation reaction progresses. In one exemplary embodiment, in one such embodiment the temperature of the first stage reaction mixture does not exceed 50° C. at least until the ratio of the number of equivalents of hydrogen reacted with β-farnesene (i.e., eq H₂:eq. β-farnesene) exceeds 0.1:1, respectively. By way of further example in one such embodiment the temperature of the first stage reaction mixture does not exceed 60° C. at least until the ratio of the number of equivalents of hydrogen reacted with β-farnesene (i.e., eq H₂:eq. β-farnesene) exceeds 0.2:1, respectively. By way of further example in one such embodiment the temperature of the first stage reaction mixture does not exceed 80° C. at least until the ratio of the number of equivalents of hydrogen reacted with β-farnesene (i.e., eq H₂:eq. β-farnesene) exceeds 0.9:1, respectively. By way of further example in one such embodiment the temperature of the first stage reaction mixture does not exceed 100° C. at least until the ratio of the number of equivalents of hydrogen reacted with β-farnesene (i.e., eq H₂:eq. β-farnesene) exceeds 0.8:1, respectively. In each of these embodiments, the temperature of the first stage reaction mixture be allowed to increase, in some embodiments to temperatures in excess of 100° C. (e.g., 110° C., 120° C. or even greater temperatures) after the ratio of the number of equivalents of hydrogen reacted with β-farnesene (i.e., eq H₂:eq. β-farnesene) exceeds 0.9:1, 1:1 or 1.1:1, respectively. In one illustrative embodiment, hydrogen is introduced at flow rate of 0.0045 kg H₂/kg β-farnesene-hr to a stage 1 reactor containing β-farnesene and hydrogenation catalyst mixture at room temperature. As soon as stirring is started and after about 0.1 molar equivalent of H₂ added, the exotherm from the hydrogenation reaction will heat the system to 60° C. After 0.2 molar equivalents of H₂ are added, heat is removed from the reaction mixture to maintain the reaction temperature at 80° C. until 1.2 molar equivalent of H₂ are added.

In one embodiment, the concentration of β-farnesene in the first stage reaction mixture is relatively dilute to favor the hydrogenation of β-farnesene over the formation of β-farnesene derivatives having a molecular weight greater than farnesane. In general, the amount of liquid diluent combined with β-farnesene to form the first stage reaction mixture may vary over a wide range. For example, in one embodiment the relative amount of diluent and β-farnesene combined to form the first stage reaction mixture may be in the range of about 1:100 to about 10:1 β-farnesene:liquid diluent. By way of further example, in one such embodiment the relative amounts of diluent and β-farnesene introduced to combined to form the first stage reaction mixture may be at least about 1:100, 1:50, 1:20, 1:10, 1:5, 1:4, 1:3, 1:2, 1:1, 2:1, 3:1, 4:1, or even about 5:1 (β-farnesene:liquid diluent). In general, however, in each such embodiment the relative amounts of diluent and β-farnesene introduced to combined to form the first stage reaction mixture will typically be less than about 10:1 (β-farnesene:liquid diluent).

In one embodiment, the liquid diluent is partially hydrogenated β-farnesene. For example, in one such embodiment, partially hydrogenated β-farnesene is removed from a reaction vessel in which β-farnesene and/or partially hydrogenated β-farnesene is being hydrogenated (e.g., a first stage and/or a second stage hydrogenation reactor as described elsewhere herein) and recycled to a reaction vessel in which the first stage of hydrogenation is being carried out. In one such embodiment the relative amount of partially hydrogenated β-farnesene diluent and β-farnesene combined to form the first stage reaction mixture may be in the range of about 1:100 to about 10:1 β-farnesene:partially hydrogenated β-farnesene. By way of further example, in one such embodiment the relative amounts of partially hydrogenated β-farnesene diluent and β-farnesene introduced to combined to form the first stage reaction mixture may be at least about 1:100, 1:50, 1:20, 1:10, 1:5, 1:4, 1:3, 1:2, 1:1, 2:1, 3:1, 4:1, or even about 5:1 (β-farnesene: partially hydrogenated β-farnesene diluent). In general, however, in each such embodiment the relative amounts of partially hydrogenated β-farnesene diluent and β-farnesene introduced to combined to form the first stage reaction mixture will typically be less than about 10:1 (β-farnesene: partially hydrogenated β-farnesene diluent).

In one embodiment, the liquid diluent comprises any of a wide range of diluents that may be easily separated from the product, e.g., by distillation. Thus, for example, in some embodiments, the liquid diluent may have a higher boiling point than the β-farnesene, such as a high boiling PAO (e.g., Durasyn® PAOs, such as Durasyn® 164, available from Ineos Oligomers, League City, Tex.), or may comprise a higher boiling oil (e.g., squalane). By way of further example, in one such embodiment, the liquid diluent comprises partially hydrogenated β-farnesene and any of a wide range of diluents that may be easily separated from the product, e.g., by distillation. In each of these embodiments, the relative amount of diluent and β-farnesene combined to form the first stage reaction mixture may be in the range of about 1:100 to about 10:1 β-farnesene:liquid diluent.

The catalyst type (and associated catalysis conditions) for the first stage hydrogenation reaction are selected to be those that are known in the art to be selective for hydrogenating conjugated diene moieties and are active at temperatures below which thermal dimerization, cyclization, isomerization, or other competing or degradation process of the conjugated alkene occurs. For example, a catalyst system that is active at a temperature in a range from about 20° C. to about 120° C. may be used to catalyze hydrogenation of β-farnesene for a first stage to reduce probability that such a competing process occurs. Exemplary first stage hydrogenation catalysts include palladium, platinum, nickel, copper, copper-chromium, rhodium, ruthenium or molybdenum on any of a range of supports may be used in the first stage. For example, in one such embodiment the catalyst may comprise nickel (e.g., about 8-21 wt % of the supported catalyst), palladium (about 0.1-0.6 wt % of the supported catalyst), or a combination of palladium and silver (about 0.5 wt % palladium and about 0.2 wt % silver of the supported catalyst. By way of further example, in one such embodiment, exemplary catalysts include palladium on an alumina support (e.g., 0.1 wt %-0.6 wt % palladium on alumina), palladium on a carbon support (e.g., 0.5 wt % palladium on carbon), and palladium/silver on an alumina support (e.g., 0.3 wt % Pd and 0.15 wt % silver on alumina). The catalyst may be in the shape of spheres, tables, extrudates, and trilobe extrudates. In one embodiment, the catalyst is recycled to the first stage reactor for improved economics.

In general, it is desired to deliver a controlled amount of hydrogen under controlled reaction conditions so as to control the extent of and site selectivity of the hydrogenation in the first stage. Typically, the β-farnesene will be reacted with about 0.8-1.5 equivalents of hydrogen per equivalent of β-farnesene in the first stage. For example, in one embodiment the β-farnesene is reacted with about 0.9-1.4 equivalents of hydrogen per equivalent of β-farnesene in the first stage. By way of further example, the β-farnesene will be reacted with about 0.9-1.3 equivalents of hydrogen per equivalent of β-farnesene in the first stage. By way of further example, the β-farnesene will be reacted with about 1-1.3 equivalents of hydrogen per equivalent of β-farnesene in the first stage. By way of further example, the β-farnesene will be reacted with about 1-1.2 equivalents of hydrogen per equivalent of β-farnesene in the first stage. By way of further example, the β-farnesene will be reacted with about 1.1-1.2 equivalents of hydrogen per equivalent of β-farnesene in the first stage.

The hydrogen pressure in the first stage may be within a wide range. In general, however, hydrogen pressures will typically be in the range of about 10 psig to about 100 psig. For example, in one embodiment the hydrogen pressure in the first stage will be about 20, 30, 40, or 50 psig. In general, hydrogen pressures in excess of about 50 psig do not provide a significant advantage.

The hydrogenation of β-farnesene is a highly exothermic reaction. Thus, the hydrogen uptake rate in the first stage will typically be limited by the cooling required to stay within the target temperature ranges for the first stage. In general, however, the hydrogen uptake rate will be in the range of about 0.002 to about 0.02 kg H₂/kg β-farnesene feed-hr. For example, in one embodiment the hydrogen uptake rate will be in the range of about 0.004 to about 0.009 kg H₂/kg β-farnesene feed-hr for reactors having a capacity in the range of 1,000 to 10,000 gallons.

Referring again to FIG. 1, in the second stage S2 of the hydrogenation process, the first stage reaction product 16 (containing partially hydrogenated β-farnesene) is reacted with hydrogen 14 in the presence of a second stage catalyst to form second stage reaction product 18 as previously described. For example, process reaction conditions may be adjusted in the second stage to convert dihydro-β-farnesene and tetrahydro-β-farnesene (in the first stage reaction product 16) to hexahydro-β-farnesene without significant concern for the formation of β-farnesene derivatives having a molecular weight greater than farnesane. Thus, for example, the second stage of the hydrogenation process may be operated at a significantly greater temperature than the first stage. In one embodiment, the second stage is operated a temperature in excess of about 120° C. In one such embodiment, the second stage is operated at a temperature of at least 200° C., at least 210° C., at least 220° C., at least 230° C., at least 240° C., at least 250° C., or even at least 260° C., In general, however, the second stage will be operated at a temperature of less than 300° C. Thus, in some embodiments the second stage of the hydrogenation process will be operated at a temperature in the range of about 240° C. to about 300° C., or even about 240° C. to about 280° C. Typically, the temperature will be increased to the second stage reaction temperature as rapidly as is practical. For example, the temperature of the reaction mixture in vessel S2 may be rapidly increased from the temperature of first stage reaction product 16 to a temperature in the range of about 200-300° C. as rapidly as possible.

The hydrogenation catalyst for the second stage hydrogenation reaction may be any of a wide range of conventional hydrogenation catalysts known in the art for selectively hydrogenating di-olefins or polyenes to produce mono-olefins. Exemplary second stage hydrogenation catalysts include palladium, platinum, nickel, copper, copper-chromium, rhodium, ruthenium or molybdenum on any of a range of supports. For example, in one such embodiment the second stage catalyst may comprise nickel (e.g., about 8-21 wt % of the supported catalyst), palladium (about 0.1-0.6 wt % of the supported catalyst), or a combination of palladium and silver (about 0.5 wt % palladium and about 0.2 wt % silver of the supported catalyst. By way of further example, in one such embodiment, exemplary catalysts include an alumina support (e.g., 0.1 wt %-0.6 wt % palladium on alumina), palladium on a carbon support (e.g., 0.5 wt % palladium on carbon), palladium/silver on an alumina support (e.g., 0.3 wt % Pd and 0.15 wt % silver on alumina), or palladium on a titanium silicate, silica, titania, zirconia or alumina-silica support. The catalyst may be in the shape of spheres, tables, extrudates, and trilobe extrudates. In one such embodiment, the second stage hydrogenation catalyst is the same catalyst as the hydrogenation catalyst for the first stage hydrogenation reaction. In another such embodiment, the second stage hydrogenation catalyst is a different catalyst system than the first stage catalyst system. In one embodiment, the catalyst is recycled to the second stage reactor for improved economics.

By maintaining the hydrogen flow rate in the range of 0.002 to about 0.02 kg H₂/kg β-farnesene feed-hr the hydrogen pressure in the second stage is not critical to selectivity and is only required to ensure adequate hydrogen uptake rate and prevent catalyst deactivation. The H₂ pressure does not need to be reduced or adjusted to control selectivity or product distribution.

The hydrogen pressure in the second stage will typically be in a range from about 10 psig-100 psig. For example, in one embodiment the hydrogen pressure in the second stage will be about 20, 30, 40, or 50 psig. In general, hydrogen pressures in excess of about 70 psig do not provide a significant advantage.

Any suitable configuration for staged partial hydrogenation may be used to carry out the methods described herein. The catalysis conditions (structure of catalyst, type of catalyst, catalyst loading, reaction time, temperature and/or hydrogen pressure) of the first stage, second stage (and subsequent stages, if present) may be independently varied. In some variations, the hydrogenation may be conducted in a single reactor such that the catalyst is not changed between stages. In some variations, the hydrogenation may be conducted in two or more reactors, configured serially, so that the catalyst used in different stages may be different. In some variations the hydrogenation product stream is split and a portion of the split stream is sent back to the reactor to enhance the reactor performance e.g. to enhance the degree of mixing, or manage the heat of reaction. If a single reactor is used for a multi-stage hydrogenation, a batch reactor (e.g., batch slurry reactor) or fixed bed or flow-through type reactor may be used. If a batch reactor is used, any suitable type of batch reactor may be used, e.g., a batch slurry reactor.

If a fixed bed or flow-through reactor, any suitable type of fixed bed or flow-through type reactor may be used. In a flow-through reaction, efficient heat transfer to the β-farnesene and residence time in certain temperature zones are important for effective staged hydrogenation reaction to achieve desired selective hydrogenation as described herein. The reactor operates safely while removing exothermic heat due to the hydrogenation, and while controlling temperature in the desired ranges. In some variations, diameters of fixed bed reactors are limited to allow control of the exotherm and overall temperature control of the reactor.

The multiple stage hydrogenation as described herein may be adapted to a variety of different reactor configurations. In some variations, multiple catalyst beds are used with interstage coolers. In some variations, a multiple tube reactor is used. In some variations, a continuous slurry reactor is used. In some variations, a fluidized bed reactor is used.

In some variations, multiple hydrogenation stages are configured as multiple zones in a fixed bed reactor. One non-limiting example of a multi-stage hydrogenation process is one in which the reactor is a flow-through reactor such as a plug flow reactor. If multiple reactors are used in a multi-stage hydrogenation process, any combination of batch reactors and fixed bed or flow-through type reactors may be used.

In some variations, the multi-stage hydrogenation is carried out in a batch reactor (e.g., batch slurry reactor), or in a series of batch reactors, wherein one or more stages (e.g., a first stage) is carried out in a first batch reactor and one or more subsequent stages (e.g., a second stage) is carried out in a second batch reactor, and so on. In some variations, at least one stage (e.g., a first stage or a second stage) of a multi-stage hydrogenation process is carried out in a fixed bed or flow-through type reactor, such as a plug-flow reactor. In some variations, more than one stage (e.g., each of the stages) of a multi-stage hydrogenation is carried out in a fixed bed or flow-through type reactor. In some variations, a first stage of a multi-stage hydrogenation is carried out in a fixed bed or flow-through type reactor and a second or subsequent stage is carried out in a batch reactor. In some variations, a first stage of a multi-stage hydrogenation is carried out in a batch reactor and a second or subsequent stage is carried out in a fixed bed or flow-through type reactor.

In one exemplary embodiment, the first and second stages are carried out in any of the various reactor configurations described elsewhere herein and the temperature of the first stage reaction mixture and the second stage reaction mixture is allowed to increase as a function of the extent of reaction (measured as a ratio of the number of equivalents of hydrogen (H₂) reacted with β-farnesene) as set forth in Table III. In general, this temperature profile (relative to certain other temperature profiles described herein) tends to provide lesser amounts of β-farnesene derivatives having a molecular weight greater than farnesane (e.g., β-farnesene dimer and polymer byproducts) and an increased reaction rate to provide greater throughput or shorter batch cycle times.

TABLE III

| | Reaction Mixture | Extent of Reaction |
|---|---|---|
| | Temp | (eq. H₂ to eq. β- |
| | (° C.) | farnesene) |
| Stage I | ≦100 | 0.0 |
| | ≦100 | 0.1 |
| | ≦100 | 0.2 |
| | ≦100 | 0.5 |
| | ≦100 | 0.8 |
| | ≦120 | 0.9 |
| | ≦120 | 1.0 |
| | ≦160 | 1.1 |
| Stage II | ≦260 | 1.2 |
| | ≦310 | 1.3 |
| | ≦310 | 3.1 |

In another exemplary embodiment, the first and second stages are carried out in any of the various reactor configurations described elsewhere herein and the temperature of the first stage reaction mixture and the second stage reaction mixture is allowed to increase as a function of the extent of reaction (measured as a ratio of the number of equivalents of hydrogen (H₂) reacted with β-farnesene) as set forth in Table IV. In general, in this embodiment the temperature profile (as compared to the temperature profile of certain other exemplary embodiments described herein) provides less operation flexibility, but reduces the formation of β-farnesene derivatives having a molecular weight greater than farnesane (e.g., β-farnesene dimer and polymer byproducts); for example, it may improve selectivity and decrease residual unhydrogenated β-farnesene resulting from due to increased residence time distribution, axial mixing, diffusion limitations or other non-ideal reactor or reaction characteristics in the first stage.

TABLE IV

| | Reaction Mixture | Extent of Reaction |
|---|---|---|
| | Temp | (eq. H₂ to eq. β- |
| | (° C.) | farnesene) |
| Stage I | ≦80 | 0.0 |
| | ≦80 | 0.1 |
| | ≦80 | 0.2 |
| | ≦80 | 0.5 |
| | ≦80 | 0.8 |
| | ≦80 | 0.9 |
| | ≦100 | 1.0 |
| | ≦120 | 1.1 |
| Stage II | ≦260 | 1.2 |
| | ≦260 | 1.3 |
| | ≦300 | 1.4 |
| | ≦300 | 3.1 |

In another exemplary embodiment, the first and second stages are carried out in any of the various reactor configurations described elsewhere herein and the temperature of the first stage reaction mixture and the second stage reaction mixture is allowed to increase as a function of the extent of reaction (measured as a ratio of the number of equivalents of hydrogen (H₂) reacted with β-farnesene) as set forth in Table V. In general, in this embodiment the temperature profile (relative to the temperature profile of certain other exemplary embodiments disclosed herein) further minimizes the formation of β-farnesene derivatives having a molecular weight greater than farnesane (e.g., β-farnesene dimer and polymer byproducts) but requires considerable cooling capability and/or greater initial hydrogen pressure to remove the heat of reaction and maintain the reaction temperature without reducing the reaction rate relative to the embodiments described in Tables III and IV.

TABLE V

| | Reaction Mixture | Extent of Reaction |
|---|---|---|
| | Temp | (eq. H₂ to eq. β- |
| | (° C.) | farnesene) |
| Stage I | ≦30 | 0.0 |
| | ≦50 | 0.1 |
| | ≦60 | 0.2 |
| | ≦80 | 0.5 |
| | ≦80 | 0.9 |
| | ≦80 | 1.0 |
| | ≦100 | 1.1 |
| | ≦120 | 1.2 |
| Stage II | ≦260 | 1.3 |
| | ≦310 | 1.4 |
| | ≦310 | 3.1 |

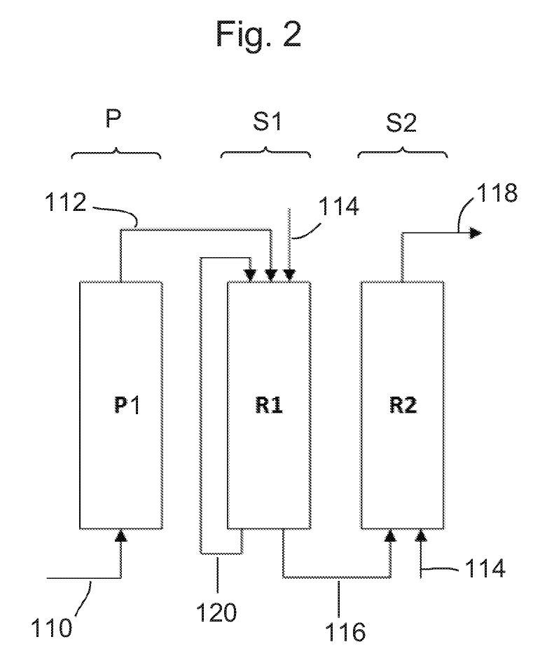

Referring now to FIG. 2, in one alternative embodiment of the present disclosure, feed stream 110 containing β-farnesene starting material is pretreated in pretreatment reaction vessel P1 to remove impurities such as acids, alcohols and/or epoxides in the β-farnesene that can poison or deactivate the hydrogenation catalyst(s) in downstream operations. Pretreated β-farnesene feed stream 112, hydrogen 114 and hydrogenation catalyst (not shown) are combined to form a first stage reaction mixture (not shown) in reaction vessel R1 and reacted to produce a first stage reaction product stream 116 as previously described. First stage reaction product stream 116, hydrogen 114 and hydrogenation catalyst (not shown) are combined in reaction vessel R2 to form a second stage reaction mixture (not shown) in reaction vessel R2 and reacted to produce second stage reaction product stream 118 as previously described. In this embodiment, reaction vessel R1 includes a recycle loop 120 to recycle and reintroduce first stage reaction product to reaction vessel R1.

Advantageously, recycling significant amounts of the first stage reaction product to reaction vessel R1 enables greater control over the temperature of the first stage reaction mixture and the concentration of β-farnesene in the first stage reaction mixture without significantly increasing the concentration of farnesane and β-farnesene derivatives having a molecular weight greater than farnesane in second stage reaction product stream 118. In one embodiment, the recycle rate, i.e., the volumetric ratio of stream 120 to stream 112 is significant. For example, the recycle rate may be 1:1, 2:1, 5:1, 10:1, 25:1, 50:1, 100:1, 500:1, or even at least 900:1 (volumetric ratio of stream 120 to stream 112, respectively).

In one exemplary embodiment, the first stage reaction is carried out in the liquid phase. Thus, for example, reaction vessel R1 may be a liquid phase plug flow reactor that is operated adiabatically, isothermally, or partially adiabatically and partially isothermally.

In one exemplary embodiment, the second stage reaction is carried out in the vapor phase. Thus, for example, reaction vessel R2 may be a vapor phase plug flow reactor that is operated adiabatically, isothermally, or partially adiabatically and partially isothermally.

In one alternative embodiment of the present disclosure, in lieu of or in addition to recycle loop 120, diluent may be introduced to reaction vessel R1 (for example, via an additional feed stream or by introduction into feed stream 112 upstream of reaction vessel R1) to provide greater control over the temperature of the first stage reaction mixture and the concentration of β-farnesene in the first stage reaction mixture without significantly increasing the concentration of farnesane and β-farnesene derivatives having a molecular weight greater than farnesane in second stage reaction product stream 118. Exemplary diluents include any of the previously identified solvents that may be readily separated from the product. For example, in some embodiments the diluent has a boiling point that is less than the boiling point of farnesane. Exemplary low boiling solvents include hexane, heptane, iso-octane and isoparaffins. The amount of diluent may be significant. For example, the volumetric ratio of diluent introduced to reaction vessel R1 to β-farnesene in stream 112 may be at least 1:1, 2:1, 5:1, 10:1, 25:1, or at least 50:1.

In one alternative embodiment of the present disclosure, diluent may be introduced to reaction vessel R2 (for example, via an additional feed stream or by introduction into stream 116 upstream of reaction vessel R2) to provide greater control over the temperature of the second first reaction mixture. In this embodiment, diluent may be introduced to reaction vessel R2 in addition to the introduction of diluent into reaction vessel R1 as previously described. Exemplary diluents include any of the previously identified solvents that may be readily separated from the product. For example, in some embodiments the diluent has a boiling point that is less than the boiling point for farnesane. Exemplary low boiling solvents include hexane, heptane, iso-octane and isoparaffins. The amount of diluent may be significant. For example, the volumetric ratio of diluent introduced to reaction vessel R1 to β-farnesene in stream 116 (in addition to any diluent that may carried over to reaction vessel R2 from reaction vessel R1) may be at least 1:1, 2:1, 5:1, 10:1, 25:1, or at least 50:1.

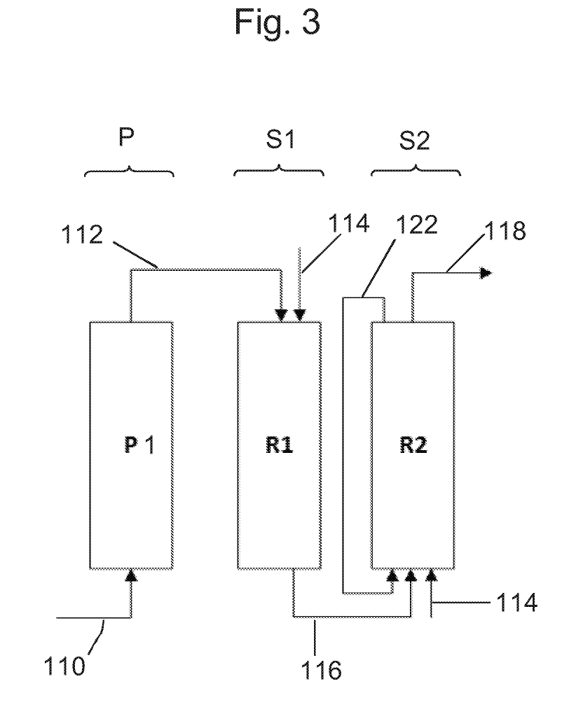

Referring now to FIG. 3, in one alternative embodiment of the present disclosure, feed stream 110 containing β-farnesene starting material is pretreated in pretreatment reaction vessel P1 to remove impurities such as acids, alcohols and/or epoxides in the β-farnesene that can poison or deactivate the hydrogenation catalyst(s) in downstream operations. Pretreated β-farnesene feed stream 112, hydrogen 114 and hydrogenation catalyst (not shown) are combined in reaction vessel R1 to form a first stage reaction mixture (not shown) in reaction vessel R1 and reacted to produce a first stage reaction product stream 116 as previously described. First stage reaction product stream 116, hydrogen 114 and hydrogenation catalyst (not shown) are combined in reaction vessel R2 to form a second stage reaction mixture (not shown) in reaction vessel R2 and reacted to produce second stage reaction product stream 118 as previously described. In this embodiment, reaction vessel R2 includes a recycle loop 122 to recycle and reintroduce second stage reaction product to reaction vessel R2. In one embodiment, the recycle rate, i.e., the volumetric ratio of stream 122 to stream 118 is significant. For example, the recycle rate may be at least 1:1, 2:1, 5:1, 10:1, 25:1, 50:1, 100:1, 500:1, or at least 900:1 (volumetric ratio of stream 122 to stream 118, respectively).

In one alternative embodiment of the present disclosure, diluent may be introduced to reaction vessel R2 (for example, via an additional feed stream or by introduction into stream 116 upstream of reaction vessel R2) to provide greater control over the temperature of the second first reaction mixture. In this embodiment, diluent may be introduced to reaction vessel R2 in addition to the introduction of diluent into reaction vessel R1 as previously described in connection with FIG. 2. Exemplary diluents include any of the previously identified solvents that may be readily separated from the product. For example, in some embodiments the diluent has a boiling point that is less than the boiling point for farnesane. Exemplary low boiling solvents include hexane, heptane, iso-octane and isoparaffins. Again, the amount of diluent may be significant. For example, the volumetric ratio of diluent introduced to reaction vessel R2 to partially hydrogenated β-farnesene in stream 116 (in addition to any diluent that may carried over to reaction vessel R2 from reaction vessel R1) may be at least 1:1, 2:1, 5:1, 10:1, 25:1, or at least 50:1.

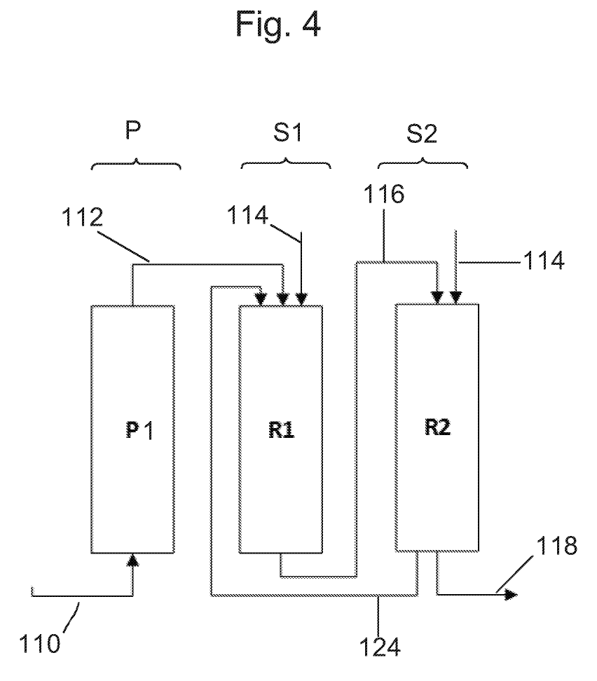

Referring now to FIG. 4, in one alternative embodiment of the present disclosure, feed stream 110 containing β-farnesene starting material is pretreated in pretreatment reaction vessel P1 to remove impurities such as acids, alcohols and/or epoxides in the β-farnesene that can poison or deactivate the hydrogenation catalyst(s) in downstream operations. Pretreated β-farnesene feed stream 112, hydrogen 114 and hydrogenation catalyst (not shown) are combined in reaction vessel R1 to form a first stage reaction mixture (not shown) in reaction vessel R1 and reacted to produce a first stage reaction product stream 116 as previously described. First stage reaction product stream 116, hydrogen 114 and hydrogenation catalyst (not shown) are combined in reaction vessel R2 to form a second stage reaction mixture (not shown) in reaction vessel R2 and reacted to produce second stage reaction product stream 118 as previously described. In this embodiment, reaction vessel R1 includes a recycle loop 124 to recycle second stage reaction product to reaction vessel R1. Advantageously, recycling significant amounts of the second stage reaction product to reaction vessel R1 enables greater control over the temperature of the first stage reaction mixture and the concentration of β-farnesene in the first stage reaction mixture without significantly increasing the concentration of farnesane and β-farnesene derivatives having a molecular weight greater than farnesane in second stage reaction product stream 118. In one embodiment, the recycle rate, i.e., the volumetric ratio of stream 124 to stream 112 is significant. For example, the recycle rate may be 1:1, 2:1, 5:1, 10:1, 25:1, 50:1, 100:1, 500:1, or even at least 900:1 (volumetric ratio of stream 124 to stream 112, respectively).

In one alternative embodiment of the present disclosure, in lieu of or in addition to recycle loop 124, diluent may be introduced to reaction vessel R1 (for example, via an additional feed stream or by introduction into feed stream 112 upstream of reaction vessel R1) to provide greater control over the temperature of the first stage reaction mixture as otherwise previously described in connection with FIG. 2. Exemplary diluents include any of the previously identified solvents that may be readily separated from the product. For example, in some embodiments the diluent has a boiling point that is less than the boiling point for farnesane. Exemplary low boiling solvents include hexane, heptane, iso-octane and isoparaffins. The amount of diluent may be significant. For example, the volumetric ratio of diluent introduced to reaction vessel R1 to β-farnesene in stream 112 may be at least 1:1, 2:1, 5:1, 10:1, 25:1, or at least 50:1.

In one alternative embodiment of the present disclosure, diluent may be introduced to reaction vessel R2 (for example, via an additional feed stream or by introduction into stream 116 upstream of reaction vessel R2) to provide greater control over the temperature of the second first reaction mixture. In this embodiment, diluent may be introduced to reaction vessel R2 in addition to the introduction of diluent into reaction vessel R1 as previously described. Exemplary diluents include any of the previously identified solvents that may be readily separated from the product. For example, in some embodiments the diluent has a boiling point that is less than the boiling point for farnesane. Exemplary low boiling solvents include hexane, heptane, iso-octane and isoparaffins. The amount of diluent may be significant. For example, the volumetric ratio of diluent introduced to reaction vessel R2 to partially hydrogenated β-farnesene in stream 116 (in addition to any diluent that may carried over to reaction vessel R2 from reaction vessel R1) may be at least 1:1, 2:1, 5:1, 10:1, 25:1, or at least 50:1.

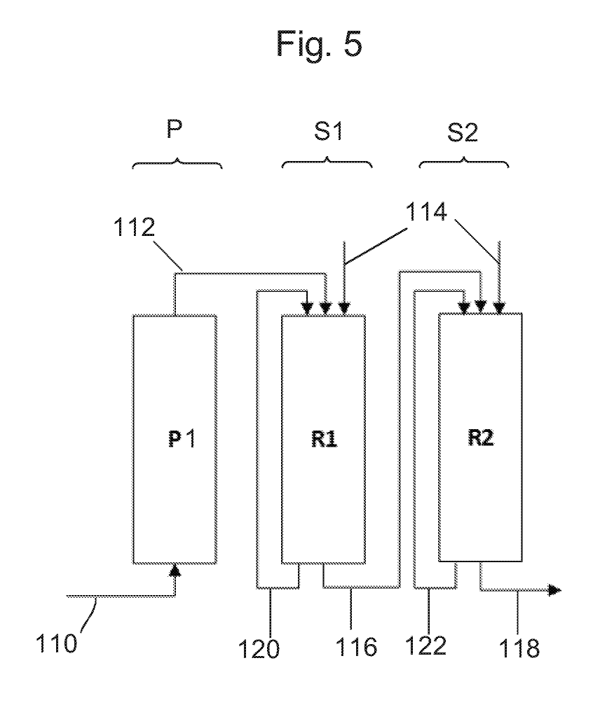

Referring now to FIG. 5, in one alternative embodiment of the present disclosure, feed stream 110 containing β-farnesene starting material is pretreated in pretreatment reaction vessel P1 to remove impurities such as acids, alcohols and/or epoxides in the β-farnesene that can poison or deactivate the hydrogenation catalyst(s) in downstream operations. Pretreated β-farnesene feed stream 112, hydrogen 114 and hydrogenation catalyst (not shown) are combined in reaction vessel R1 to form a first stage reaction mixture (not shown) in reaction vessel R1 and reacted to produce a first stage reaction product stream 116 as previously described. First stage reaction product stream 116, hydrogen 114 and hydrogenation catalyst (not shown) are combined in reaction vessel R2 to form a second stage reaction mixture (not shown) in reaction vessel R2 and reacted to produce second stage reaction product stream 118 as previously described. In this embodiment, reaction vessel R1 includes a recycle loop 124 to recycle second stage reaction product to reaction vessel R1 and reaction vessel R2 includes a recycle loop 122 to recycle and reintroduce second stage reaction product to reaction vessel R2. In one embodiment, the recycle rate, i.e., the volumetric ratio of stream 124 to stream 112 is significant. For example, the recycle rate may be 1:1, 2:1, 5:1, 10:1, 25:1, 50:1, 100:1, 500:1, or even at least 900:1 (volumetric ratio of stream 124 to stream 112, respectively). Additionally, in each of these embodiments, the recycle rate, i.e., the volumetric ratio of stream 122 to stream 118 is significant. For example, the recycle rate may be at least 1:1, 2:1, 5:1, 10:1, 25:1, 50:1, 100:1, 500:1, or at least 900:1 (volumetric ratio of stream 122 to stream 118, respectively)

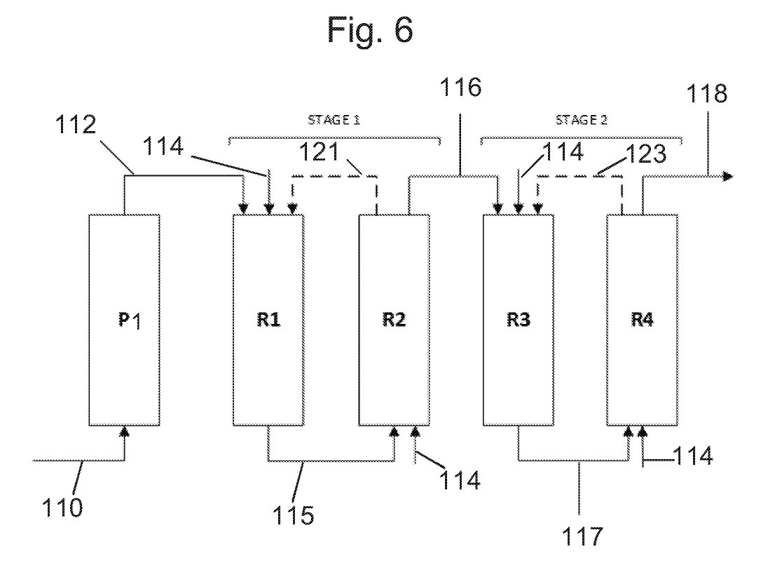

Referring now to FIG. 6, in one alternative embodiment of the present disclosure, feed stream 110 containing β-farnesene starting material is pretreated in pretreatment reaction vessel P1 to remove impurities such as acids, alcohols and/or epoxides in the β-farnesene that can poison or deactivate the hydrogenation catalyst(s) in downstream operations. Pretreated β-farnesene feed stream 112, hydrogen 114 and hydrogenation catalyst (not shown) are combined in reaction vessel R1 to form a first intermediate first stage reaction mixture (not shown) in reaction vessel R1 and reacted to produce an intermediate first stage reaction product stream 115. Intermediate first stage reaction product stream 115 is combined with hydrogen 114 and hydrogenation catalyst (not shown) in reaction vessel R2 to form a second intermediate first stage reaction mixture (not shown) in reaction vessel R2 and reacted to produce first stage reaction product 116 as previously described. First stage reaction product stream 116, hydrogen 114 and hydrogenation catalyst (not shown) are combined in reaction vessel R3 to form a first intermediate second stage reaction mixture (not shown) in reaction vessel R3 and reacted to produce intermediate second stage reaction product stream 117. Intermediate second stage reaction product stream 117 is combined with hydrogen 114 and hydrogenation catalyst (not shown) in reaction vessel R4 to form a second intermediate second stage reaction mixture (not shown) in reaction vessel R4 and reacted to produce second stage reaction product 118 as previously described. In this embodiment, reaction vessel R2 includes an optional recycle loop 121 to recycle first stage reaction product 116 to reaction vessel R1 and reaction vessel R4 includes an optional recycle loop 123 to recycle second stage reaction product 118 to reaction vessel R3. In one embodiment, the recycle rate, i.e., the volumetric ratio of stream 121 to stream 112 is significant. For example, the recycle rate may be at least 1:1, 2:1, 3:1 4:1, 5:1, 6:1, 7:1, 8:1, 9:1 or even greater than 9:1 (volumetric ratio of stream 121 to stream 112, respectively). Additionally, in each of these embodiments, the recycle rate, i.e., the volumetric ratio of stream 123 to stream 118 is significant. For example, the recycle rate may be at least 1:1, 2:1, 5:1, 10:1, 25:1, 50:1, 100:1, 500:1, or even at least 900:1 (volumetric ratio of stream 123 to stream 118, respectively)

In one exemplary embodiment, the first stage reaction is carried out in the liquid phase. Thus, for example, reaction vessels R1 and R2 (as illustrated in the embodiment of FIG. 6) may be a liquid phase plug flow reactor that is operated adiabatically, isothermally, or partially adiabatically and partially isothermally.

In one exemplary embodiment, the first stage is carried out in an adiabatic reactor where the inlet stream to R1 (as illustrated in FIG. 6) may have a temperature of 30-50° C. and an outlet temperature of 70-100° C., where the outlet stream is then cooled before entering R2. This allows for temperature to be controlled between reactors.

In one exemplary embodiment, the second stage reaction is carried out in the vapor phase. Thus, for example, reaction vessels R3 and R4 (as illustrated in the embodiment of FIG. 6) may be a vapor phase plug flow reactor that is operated adiabatically, isothermally, or partially adiabatically and partially isothermally. The inlet temperature of the second stage may be from 230 to 300° C. and may contain a lower vapor pressure diluent.

In one exemplary embodiment, the feed for the second stage reaction is vaporized by means of a thin film evaporator (TFE) or wiped film evaporator (WFE) to provide a short residence time and low pressure drop and avoid unwanted thermal degradation, dimer, or oligomer formation.

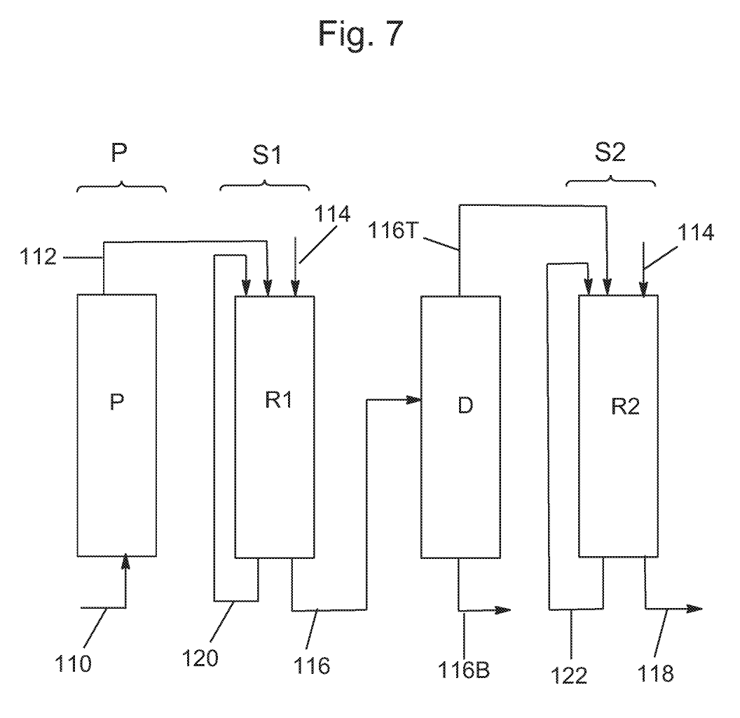

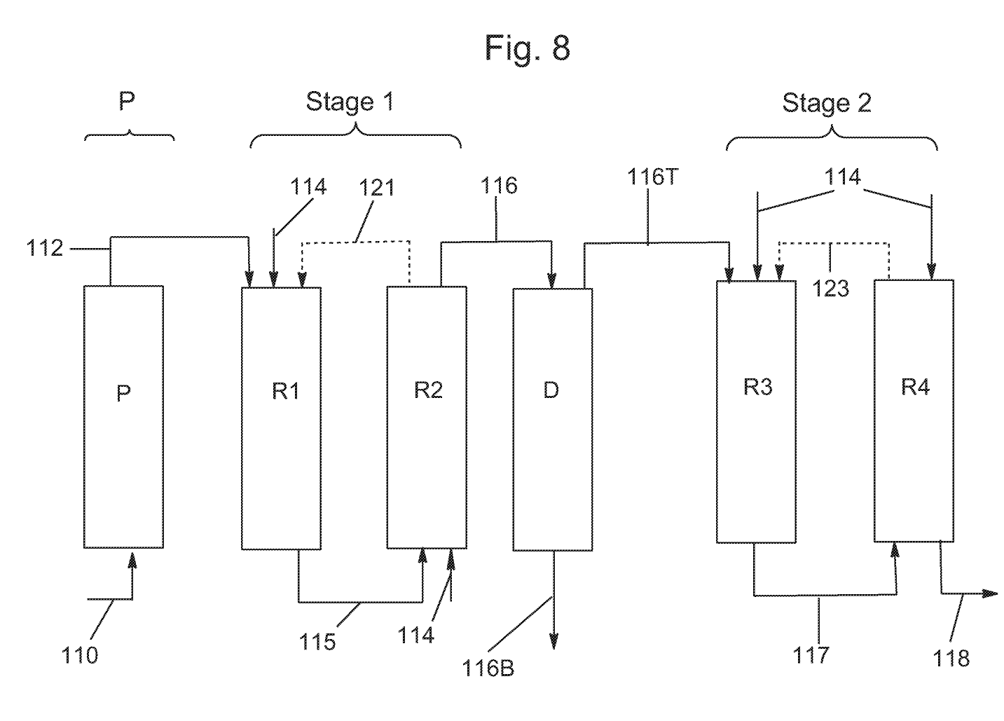

Referring now to FIGS. 7 and 8, in one alternative embodiment of the present disclosure, first stage reaction product stream 116 is separated in step D using a thin film evaporator, wiped film evaporator, distillation or other appropriate unit operation into a first stage reaction product top stream 116T and a first stage reaction product bottom stream 116B. To minimize fouling of the stage 2 (S2) catalyst bed and prolong the stage 2 (S2) catalyst life, higher molecular weight oligomers and polymer side-products present in first stage reaction product stream 116 may be separated, yielding the heavier ‘bottoms’ product as 116B a separate side-product stream and a top product 116T that is delivered to the second stage (S2) reactor. Otherwise, the embodiment illustrated in FIG. 7 corresponds to the embodiment illustrated and described in connection with FIG. 3 and the embodiment illustrated in FIG. 8 corresponds to the embodiment illustrated and described in connection with FIG. 6.

The present disclosure further includes the following enumerated embodiments.

Embodiment 1. An olefinic product comprising partially hydrogenated β-farnesene, the olefinic product having (i) a mass fraction of farnesane that is no more than 1 wt % of the combined amount of β-farnesene, partially hydrogenated β-farnesene and farnesane in the olefinic product, (ii) a mass fraction of β-farnesene that is no more than 5 wt % of the combined amount of β-farnesene, partially hydrogenated β-farnesene and farnesane in the olefinic product, and (iii) a mass fraction of β-farnesene derivatives having a molecular weight greater than farnesane that is no more than 2.5 wt % of the combined amount of β-farnesene, partially hydrogenated β-farnesene, farnesane and β-farnesene derivatives having a molecular weight greater than farnesane.

Embodiment 2. The olefinic product of enumerated Embodiment 1 wherein the mass fraction of dihydro-β-farnesene is at least 85 wt % of the combined amount of β-farnesene, partially hydrogenated β-farnesene, and farnesane in the olefinic product.

Embodiment 3. The olefinic product of enumerated Embodiment 1 wherein the mass fraction of dihydro-β-farnesene is at least 90 wt % of the combined amount of β-farnesene, partially hydrogenated β-farnesene, and farnesane in the olefinic product.

Embodiment 4. The olefinic product of enumerated Embodiment 1 wherein the mass fraction of dihydro-β-farnesene is at least 92 wt % of the combined amount of β-farnesene, partially hydrogenated β-farnesene, and farnesane in the olefinic product.

Embodiment 5. The olefinic product of enumerated Embodiment 1 wherein the mass fraction of dihydro-β-farnesene is at least 94 wt % of the combined amount of β-farnesene, partially hydrogenated β-farnesene, and farnesane in the olefinic product.

Embodiment 6. The olefinic product of enumerated Embodiment 1 wherein the mass fraction of dihydro-β-farnesene is at least 96 wt % of the combined amount of β-farnesene, partially hydrogenated β-farnesene, and farnesane in the olefinic product.

Embodiment 7. The olefinic product of any preceding enumerated

Embodiment wherein the mass fraction of partially hydrogenated β-farnesene is at least 95 wt % of the combined amount of β-farnesene, partially hydrogenated β-farnesene, and farnesane in the olefinic product.

Embodiment 8. The olefinic product of any preceding enumerated Embodiment wherein the mass fraction of partially hydrogenated β-farnesene is at least 97 wt % of the combined amount of β-farnesene, partially hydrogenated β-farnesene, and farnesane in the olefinic product.

Embodiment 9. The olefinic product of any preceding enumerated Embodiment wherein the mass fraction of partially hydrogenated β-farnesene is at least 98 wt % of the combined amount of β-farnesene, partially hydrogenated β-farnesene, and farnesane in the olefinic product.

Embodiment 10. The olefinic product of any preceding enumerated Embodiment wherein the mass fraction of partially hydrogenated β-farnesene is at least 99 wt % of the combined amount of β-farnesene, partially hydrogenated β-farnesene, and farnesane in the olefinic product.

Embodiment 11. The olefinic product of any preceding enumerated Embodiment wherein the mass fraction of β-farnesene in the olefinic product is no more than 4 wt % of the combined amount of β-farnesene, partially hydrogenated β-farnesene, and farnesane in the olefinic product.

Embodiment 12. The olefinic product of any preceding enumerated Embodiment wherein the mass fraction of β-farnesene in the olefinic product is no more than 3 wt % of the combined amount of β-farnesene, partially hydrogenated β-farnesene, and farnesane in the olefinic product.

Embodiment 13. The olefinic product of any preceding enumerated Embodiment wherein the mass fraction of β-farnesene in the olefinic product is no more than 2 wt % of the combined amount of β-farnesene, partially hydrogenated β-farnesene, and farnesane in the olefinic product.

Embodiment 14. The olefinic product of any preceding enumerated Embodiment wherein the mass fraction of β-farnesene in the olefinic product is no more than 1 wt % of the combined amount of β-farnesene, partially hydrogenated β-farnesene, and farnesane in the olefinic product.

Embodiment 15. The olefinic product of any preceding enumerated Embodiment wherein the mass fraction of β-farnesene in the olefinic product is no more than 0.5 wt % of the combined amount of β-farnesene, partially hydrogenated β-farnesene, and farnesane in the olefinic product.

Embodiment 16. The olefinic product of any preceding enumerated Embodiment wherein the mass fraction of β-farnesene in the olefinic product is no more than 0.25 wt % of the combined amount of β-farnesene, partially hydrogenated β-farnesene, and farnesane in the olefinic product.

Embodiment 17. The olefinic product of any preceding enumerated Embodiment wherein the mass fraction of β-farnesene in the olefinic product is no more than 0.1 wt % of the combined amount of β-farnesene, partially hydrogenated β-farnesene, and farnesane in the olefinic product.

Embodiment 18. The olefinic product of any preceding enumerated Embodiment wherein β-farnesene is undetectable in the olefinic product.

Embodiment 19. The olefinic product of any preceding enumerated Embodiment wherein the mass fraction of farnesane in the olefinic product is no more than 0.75 wt % of the combined amount of β-farnesene, partially hydrogenated β-farnesene, and farnesane in the olefinic product.

Embodiment 20. The olefinic product of any preceding enumerated Embodiment wherein the mass fraction of farnesane in the olefinic product is no more than 0.5 wt % of the combined amount of β-farnesene, partially hydrogenated β-farnesene, and farnesane in the olefinic product.

Embodiment 21. The olefinic product of any preceding enumerated Embodiment wherein the mass fraction of farnesane in the olefinic product is no more than 0.25 wt % of the combined amount of β-farnesene, partially hydrogenated β-farnesene, and farnesane in the olefinic product.

Embodiment 22. The olefinic product of any preceding enumerated Embodiment wherein the mass fraction of farnesane in the olefinic product is no more than 0.1 wt % of the combined amount of β-farnesene, partially hydrogenated β-farnesene, and farnesane in the olefinic product.

Embodiment 23. The olefinic product of any preceding enumerated Embodiment wherein the mass fraction of farnesane in the olefinic product is no more than 0.05 wt % of the combined amount of β-farnesene, partially hydrogenated β-farnesene, and farnesane in the olefinic product.

Embodiment 24. The olefinic product of any preceding enumerated Embodiment wherein farnesane is undetectable in the olefinic product.

Embodiment 25. An olefinic product comprising hexahydro-β-farnesene, the olefinic product having (i) a mass fraction of farnesane that is no more than 7 wt % relative to the combined amount of β-farnesene, partially hydrogenated β-farnesene and farnesane in the second stage reaction product, (ii) a mass fraction of hexahydro-β-farnesene that is at least 85 wt % of the combined amount of β-farnesene, partially hydrogenated β-farnesene and farnesane in the second stage reaction product, and (iii) a mass fraction of β-farnesene derivatives having a molecular weight greater than farnesane that is no more than 2.5 wt % of the combined amount of β-farnesene, partially hydrogenated β-farnesene, farnesane and β-farnesene derivatives having a molecular weight greater than farnesane in the second stage reaction product.

Embodiment 26. The olefinic product of enumerated Embodiment 25 wherein the mass fraction of β-farnesene in the olefinic product is no more than 0.05 wt % of the combined amount of β-farnesene, partially hydrogenated β-farnesene, and farnesane in the olefinic product.

Embodiment 27. The olefinic product of enumerated Embodiment 25 wherein β-farnesene is undetectable in the olefinic product.

Embodiment 28. The olefinic product of any of Embodiments 25 to 27 wherein the mass fraction of hexahydro-β-farnesene is at least 87 wt % of the combined amount of β-farnesene, partially hydrogenated β-farnesene, and farnesane in the olefinic product.

Embodiment 29. The olefinic product of any of Embodiments 25 to 27 wherein the mass fraction of hexahydro-β-farnesene is at least 88 wt % of the combined amount of β-farnesene, partially hydrogenated β-farnesene, and farnesane in the olefinic product.

Embodiment 30. The olefinic product of any of Embodiments 25 to 27 wherein the mass fraction of hexahydro-β-farnesene is at least 89 wt % of the combined amount of β-farnesene, partially hydrogenated β-farnesene, and farnesane in the olefinic product.

Embodiment 31. The olefinic product of any of Embodiments 25 to 27 wherein the mass fraction of hexahydro-β-farnesene is at least 90 wt % of the combined amount of β-farnesene, partially hydrogenated β-farnesene, and farnesane in the olefinic product.

Embodiment 32. The olefinic product of any of Embodiments 25 to 27 wherein the mass fraction of hexahydro-β-farnesene is at least 91 wt % of the combined amount of β-farnesene, partially hydrogenated β-farnesene, and farnesane in the olefinic product.

Embodiment 33. The olefinic product of any of Embodiments 25 to 27 wherein the mass fraction of hexahydro-β-farnesene is at least 92 wt % of the combined amount of β-farnesene, partially hydrogenated β-farnesene, and farnesane in the olefinic product.

Embodiment 34. The olefinic product of any of Embodiments 25 to 27 wherein the mass fraction of hexahydro-β-farnesene is at least 93 wt % of the combined amount of β-farnesene, partially hydrogenated β-farnesene, and farnesane in the olefinic product.

Embodiment 35. The olefinic product of any of Embodiments 25 to 34 wherein the mass fraction of farnesane in the olefinic product is no more than 6 wt % of the combined amount of β-farnesene, partially hydrogenated β-farnesene, and farnesane in the olefinic product.

Embodiment 36. The olefinic product of any of Embodiments 25 to 34 wherein the mass fraction of farnesane in the olefinic product is no more than 5 wt % of the combined amount of β-farnesene, partially hydrogenated β-farnesene, and farnesane in the olefinic product.

Embodiment 37. The olefinic product of any of Embodiments 25 to 34 wherein the mass fraction of farnesane in the olefinic product is no more than 4 wt % of the combined amount of β-farnesene, partially hydrogenated β-farnesene, and farnesane in the olefinic product.

Embodiment 38. The olefinic product of any of Embodiments 25 to 34 wherein the mass fraction of farnesane in the olefinic product is no more than 3 wt % of the combined amount of β-farnesene, partially hydrogenated β-farnesene, and farnesane in the olefinic product.

Embodiment 39. The olefinic product of any of Embodiments 25 to 34 wherein the mass fraction of farnesane in the olefinic product is no more than 2 wt % of the combined amount of β-farnesene, partially hydrogenated β-farnesene, and farnesane in the olefinic product.

Embodiment 40. The olefinic product of any of Embodiments 25 to 34 wherein the mass fraction of farnesane in the olefinic product is no more than 1 wt % of the combined amount of β-farnesene, partially hydrogenated β-farnesene, and farnesane in the olefinic product.

Embodiment 41. The olefinic product of any preceding enumerated Embodiment wherein the mass fraction of β-farnesene derivatives having a molecular weight greater than farnesane is no more than 2 wt % of the combined amount of β-farnesene, partially hydrogenated β-farnesene, farnesane, and β-farnesene derivatives having a molecular weight greater than farnesane in the olefinic product.

Embodiment 42. The olefinic product of any preceding enumerated Embodiment wherein the mass fraction of β-farnesene derivatives having a molecular weight greater than farnesane is no more than 1.5 wt % of the combined amount of β-farnesene, partially hydrogenated β-farnesene, farnesane, and β-farnesene derivatives having a molecular weight greater than farnesane in the olefinic product.

Embodiment 43. The olefinic product of any preceding enumerated Embodiment wherein the mass fraction of β-farnesene derivatives having a molecular weight greater than farnesane is no more than 1 wt % of the combined amount of β-farnesene, partially hydrogenated β-farnesene, farnesane, and β-farnesene derivatives having a molecular weight greater than farnesane in the olefinic product.

Embodiment 44. The olefinic product of any preceding enumerated Embodiment wherein the mass fraction of β-farnesene derivatives having a molecular weight greater than farnesane is no more than 0.5 wt % of the combined amount of β-farnesene, partially hydrogenated β-farnesene, farnesane, and β-farnesene derivatives having a molecular weight greater than farnesane in the olefinic product.

Embodiment 45. The olefinic product of any preceding enumerated Embodiment wherein the mass fraction of β-farnesene derivatives having a molecular weight greater than farnesane is no more than 0.25 wt % of the combined amount of β-farnesene, partially hydrogenated β-farnesene, farnesane, and β-farnesene derivatives having a molecular weight greater than farnesane in the olefinic product.

Embodiment 46. The olefinic product of any preceding enumerated Embodiment wherein the mass fraction of β-farnesene derivatives having a molecular weight greater than farnesane is no more than 0.1 wt % of the combined amount of β-farnesene, partially hydrogenated β-farnesene, farnesane, and β-farnesene derivatives having a molecular weight greater than farnesane in the olefinic product.

Embodiment 47. The olefinic product of any preceding enumerated Embodiment wherein β-farnesene derivatives having a molecular weight greater than farnesane are undetectable in the olefinic product.

Embodiment 48. A process for hydrogenating β-farnesene to form an olefinic composition, the process comprising:
(a) in a first stage, reacting the β-farnesene with hydrogen in the presence of a first stage catalyst in a first stage reaction mixture while controlling the rate of hydrogenation of the β-farnesene relative to the rate of formation of β-farnesene derivatives having a molecular weight greater than farnesane to produce a first stage reaction product wherein (i) the mass fraction of farnesane in the first stage reaction product is no more than 1 wt % of the combined amount of β-farnesene, partially hydrogenated β-farnesene and farnesane in the first stage reaction product, (ii) the mass fraction of β-farnesene in the first stage reaction product is no more than 5 wt % of the combined amount of β-farnesene, partially hydrogenated β-farnesene and farnesane in the first stage reaction product, and (iii) the mass fraction of β-farnesene derivatives having a molecular weight greater than farnesane in the first stage reaction product is no more than 2.5 wt % of the combined amount of β-farnesene, partially hydrogenated β-farnesene, farnesane and β-farnesene derivatives having a molecular weight greater than farnesane in the first stage reaction product, and
(b) in a second stage, reacting the first stage reaction product with hydrogen in the presence of a second stage catalyst to produce a second stage reaction product, wherein (i) the mass fraction of farnesane in the second stage reaction product is no more than 7 wt % of the combined amount of β-farnesene, partially hydrogenated β-farnesene and farnesane in the second stage reaction product, (ii) the mass fraction of partially hexahydro-β-farnesene in the second stage reaction product is at least 85 wt % of the combined amount of β-farnesene, partially hydrogenated β-farnesene and farnesane in the second stage reaction product, and (iii) the mass fraction of β-farnesene derivatives having a molecular weight greater than farnesane in the second stage reaction product is no more than 2.5 wt % of the combined amount of β-farnesene, partially hydrogenated β-farnesene, farnesane and β-farnesene derivatives having a molecular weight greater than farnesane in the second stage reaction product.

Embodiment 49. The process of Embodiment 48 wherein the mass fraction of dihydro-β-farnesene is at least 85 wt % of the combined amount of β-farnesene, partially hydrogenated β-farnesene, and farnesane in the first stage reaction product.

Embodiment 50. The process of Embodiment 48 wherein the mass fraction of dihydro-β-farnesene is at least 90 wt % of the combined amount of β-farnesene, partially hydrogenated β-farnesene, and farnesane in the first stage reaction product.

Embodiment 51. The process of Embodiment 48 wherein the mass fraction of dihydro-β-farnesene is at least 92 wt % of the combined amount of β-farnesene, partially hydrogenated β-farnesene, and farnesane in the first stage reaction product.

Embodiment 52. The process of Embodiment 48 wherein the mass fraction of dihydro-β-farnesene is at least 94 wt % of the combined amount of β-farnesene, partially hydrogenated β-farnesene, and farnesane in the first stage reaction product.

Embodiment 53. The process of Embodiment 48 wherein the mass fraction of dihydro-β-farnesene is at least 96 wt % of the combined amount of β-farnesene, partially hydrogenated β-farnesene, and farnesane in the first stage reaction product.

Embodiment 54. The process of any of Embodiments 48 to 53 wherein the mass fraction of partially hydrogenated β-farnesene is at least 96 wt % of the combined amount of β-farnesene, partially hydrogenated β-farnesene, and farnesane in the first stage reaction product.

Embodiment 55. The process of any of Embodiments 48 to 53 wherein the mass fraction of partially hydrogenated β-farnesene is at least 97 wt % of the combined amount of β-farnesene, partially hydrogenated β-farnesene, and farnesane in the first stage reaction product.

Embodiment 56. The process of any of Embodiments 48 to 53 wherein the mass fraction of partially hydrogenated β-farnesene is at least 98 wt % of the combined amount of β-farnesene, partially hydrogenated β-farnesene, and farnesane in the first stage reaction product.

Embodiment 57. The process of any of Embodiments 48 to 53 wherein the mass fraction of partially hydrogenated β-farnesene is at least 99 wt % of the combined amount of β-farnesene, partially hydrogenated β-farnesene, and farnesane in the first stage reaction product.

Embodiment 58. The process of any of Embodiments 48 to 57 wherein the mass fraction of partially hydrogenated β-farnesene is no more than 4 wt % of the combined amount of β-farnesene, partially hydrogenated β-farnesene, and farnesane in the first stage reaction product.

Embodiment 59. The process of any of Embodiments 48 to 57 wherein the mass fraction of partially hydrogenated β-farnesene is no more than 3 wt % of the combined amount of β-farnesene, partially hydrogenated β-farnesene, and farnesane in the first stage reaction product.

Embodiment 60. The process of any of Embodiments 48 to 57 wherein the mass fraction of partially hydrogenated β-farnesene is no more than 2 wt % of the combined amount of β-farnesene, partially hydrogenated β-farnesene, and farnesane in the first stage reaction product.

Embodiment 61. The process of any of Embodiments 48 to 57 wherein the mass fraction of partially hydrogenated β-farnesene is no more than 1 wt % of the combined amount of β-farnesene, partially hydrogenated β-farnesene, and farnesane in the first stage reaction product.

Embodiment 62. The process of any of Embodiments 48 to 57 wherein the mass fraction of partially hydrogenated β-farnesene is no more than 0.5 wt % of the combined amount of β-farnesene, partially hydrogenated β-farnesene, and farnesane in the first stage reaction product.

Embodiment 63. The process of any of Embodiments 48 to 57 wherein the mass fraction of partially hydrogenated β-farnesene is no more than 0.25 wt % of the combined amount of β-farnesene, partially hydrogenated β-farnesene, and farnesane in the first stage reaction product.

Embodiment 64. The process of any of Embodiments 48 to 57 wherein the mass fraction of partially hydrogenated β-farnesene is no more than 0.1 wt % of the combined amount of β-farnesene, partially hydrogenated β-farnesene, and farnesane in the first stage reaction product.

Embodiment 65. The process of any of Embodiments 48 to 57 wherein β-farnesene is undetectable in the first stage reaction product.

Embodiment 66. The process of any of Embodiments 48 to 65 wherein the mass fraction of farnesane in the first stage reaction product is no more than 0.75 wt % of the combined amount of β-farnesene, partially hydrogenated β-farnesene, and farnesane in the first stage reaction product.

Embodiment 67. The process of any of Embodiments 48 to 65 wherein the mass fraction of farnesane in the first stage reaction product is no more than 0.5 wt % of the combined amount of β-farnesene, partially hydrogenated β-farnesene, and farnesane in the first stage reaction product.

Embodiment 68. The process of any of Embodiments 48 to 65 wherein the mass fraction of farnesane in the first stage reaction product is no more than 0.25 wt % of the combined amount of β-farnesene, partially hydrogenated β-farnesene, and farnesane in the first stage reaction product.

Embodiment 69. The process of any of Embodiments 48 to 65 wherein the mass fraction of farnesane in the first stage reaction product is no more than 0.1 wt % of the combined amount of β-farnesene, partially hydrogenated β-farnesene, and farnesane in the first stage reaction product.

Embodiment 70. The process of any of Embodiments 48 to 65 wherein the mass fraction of farnesane in the first stage reaction product is no more than 0.05 wt % of the combined amount of β-farnesene, partially hydrogenated β-farnesene, and farnesane in the first stage reaction product.

Embodiment 71. The process of any of Embodiments 48 to 70 wherein the first stage reaction product is treated to remove β-farnesene derivatives having a molecular weight greater than farnesane before the first stage reaction product is reacted with hydrogen in the presence of a second stage catalyst to produce a second stage reaction product.

Embodiment 72. The process of any of Embodiments 48 to 70 wherein the first stage reaction product is treated in a thin film evaporator, a wiped film evaporator, or a distillation column to remove β-farnesene derivatives having a molecular weight greater than farnesane before the first stage reaction product is reacted with hydrogen in the presence of a second stage catalyst to produce a second stage reaction product.

Embodiment 73. The process of any of Embodiments 48 to 72 wherein the mass fraction of β-farnesene in the second stage reaction product is no more than 0.05 wt % of the combined amount of β-farnesene, partially hydrogenated (β-farnesene, and farnesane in the second stage reaction product.

Embodiment 74. The process of any of Embodiments 48 to 72 wherein β-farnesene is undetectable in the second stage reaction product.

Embodiment 75. The process of any of Embodiments 48 to 74 wherein the mass fraction of hexahydro-β-farnesene is at least 87 wt % of the combined amount of β-farnesene, partially hydrogenated β-farnesene, and farnesane in the second stage reaction product.

Embodiment 76. The process of any of Embodiments 48 to 74 wherein the mass fraction of hexahydro-β-farnesene is at least 88 wt % of the combined amount of β-farnesene, partially hydrogenated β-farnesene, and farnesane in the second stage reaction product.

Embodiment 77. The process of any of Embodiments 48 to 74 wherein the mass fraction of hexahydro-β-farnesene is at least 89 wt % of the combined amount of β-farnesene, partially hydrogenated β-farnesene, and farnesane in the second stage reaction product.

Embodiment 78. The process of any of Embodiments 48 to 74 wherein the mass fraction of hexahydro-β-farnesene is at least 90 wt % of the combined amount of β-farnesene, partially hydrogenated β-farnesene, and farnesane in the second stage reaction product.

Embodiment 79. The process of any of Embodiments 48 to 74 wherein the mass fraction of hexahydro-β-farnesene is at least 91 wt % of the combined amount of β-farnesene, partially hydrogenated β-farnesene, and farnesane in the second stage reaction product.

Embodiment 80. The process of any of Embodiments 48 to 74 wherein the mass fraction of hexahydro-β-farnesene is at least 92 wt % of the combined amount of β-farnesene, partially hydrogenated β-farnesene, and farnesane in the second stage reaction product.

Embodiment 81. The process of any of Embodiments 48 to 74 wherein the mass fraction of hexahydro-β-farnesene is at least 93 wt % of the combined amount of β-farnesene, partially hydrogenated β-farnesene, and farnesane in the second stage reaction product.

Embodiment 82. The process of any of Embodiments 48 to 81 wherein the mass fraction of farnesane in the second stage reaction product is no more than 6 wt % of the combined amount of β-farnesene, partially hydrogenated β-farnesene, and farnesane in the second stage reaction product.

Embodiment 83. The process of any of Embodiments 48 to 81 wherein the mass fraction of farnesane in the second stage reaction product is no more than 5 wt % of the combined amount of β-farnesene, partially hydrogenated β-farnesene, and farnesane in the second stage reaction product.

Embodiment 84. The process of any of Embodiments 48 to 81 wherein the mass fraction of farnesane in the second stage reaction product is no more than 4 wt % of the combined amount of β-farnesene, partially hydrogenated β-farnesene, and farnesane in the second stage reaction product.

Embodiment 85. The process of any of Embodiments 48 to 81 wherein the mass fraction of farnesane in the second stage reaction product is no more than 3 wt % of the combined amount of β-farnesene, partially hydrogenated β-farnesene, and farnesane in the second stage reaction product.

Embodiment 86. The process of any of Embodiments 48 to 81 wherein the mass fraction of farnesane in the second stage reaction product is no more than 2 wt % of the combined amount of β-farnesene, partially hydrogenated β-farnesene, and farnesane in the second stage reaction product.

Embodiment 87. The process of any of Embodiments 48 to 81 wherein the mass fraction of farnesane in the second stage reaction product is no more than 1 wt % of the combined amount of β-farnesene, partially hydrogenated β-farnesene, and farnesane in the second stage reaction product.

Embodiment 88. The process of any of Embodiments 48 to 87 wherein the mass fraction of β-farnesene derivatives having a molecular weight greater than farnesane is no more than 2 wt % of the combined amount of β-farnesene, partially hydrogenated β-farnesene, farnesane, and β-farnesene derivatives having a molecular weight greater than farnesane in the first and second stage reaction products.

Embodiment 89. The process of any of Embodiments 48 to 87 wherein the mass fraction of β-farnesene derivatives having a molecular weight greater than farnesane is no more than 1.5 wt % of the combined amount of β-farnesene, partially hydrogenated β-farnesene, farnesane, and β-farnesene derivatives having a molecular weight greater than farnesane in the first and second stage reaction products.

Embodiment 90. The process of any of Embodiments 48 to 87 wherein the mass fraction of β-farnesene derivatives having a molecular weight greater than farnesane is no more than 1 wt % of the combined amount of β-farnesene, partially hydrogenated β-farnesene, farnesane, and β-farnesene derivatives having a molecular weight greater than farnesane in the first and second stage reaction products.

Embodiment 91. The process of any of Embodiments 48 to 87 wherein the mass fraction of β-farnesene derivatives having a molecular weight greater than farnesane is no more than 0.5 wt % of the combined amount of β-farnesene, partially hydrogenated β-farnesene, farnesane, and β-farnesene derivatives having a molecular weight greater than farnesane in the first and second stage reaction products.

Embodiment 92. The process of any of Embodiments 48 to 87 wherein the mass fraction of β-farnesene derivatives having a molecular weight greater than farnesane is no more than 0.25 wt % of the combined amount of β-farnesene, partially hydrogenated β-farnesene, farnesane, and β-farnesene derivatives having a molecular weight greater than farnesane in the first and second stage reaction products.

Embodiment 93. The process of any of Embodiments 48 to 87 wherein the mass fraction of β-farnesene derivatives having a molecular weight greater than farnesane is no more than 0.1 wt % of the combined amount of β-farnesene, partially hydrogenated β-farnesene, farnesane, and β-farnesene derivatives having a molecular weight greater than farnesane in the first and second stage reaction products.

Embodiment 94. The process of any of Embodiments 48-93 wherein the temperature of the first stage reaction mixture is increased from a temperature at or near room temperature to a maximum temperature of about 120° C. as the amount of β-farnesene decreases and the amount of partially hydrogenated β-farnesene increases in the first stage reaction mixture.

Embodiment 95. The process of any of Embodiments 48-93 wherein the temperature of the first stage reaction mixture is increased from a temperature at or near room temperature to a maximum temperature of about 100° C. as the amount of β-farnesene decreases and the amount of partially hydrogenated β-farnesene increases in the first stage reaction mixture.

Embodiment 96. The process of any of Embodiments 48-93 wherein the temperature of the first stage reaction mixture is increased from a temperature at or near room temperature to a maximum temperature in the range of about 80 to 100° C. as the amount of β-farnesene decreases and the amount of partially hydrogenated β-farnesene increases in the first stage reaction mixture.

Embodiment 97. The process of any of Embodiments 48-96 wherein the temperature of the first stage reaction mixture does not exceed 50° C. at least until the ratio of the number of equivalents of hydrogen reacted with β-farnesene, respectively, exceeds 0.1:1.

Embodiment 98. The process of any of Embodiments 48-97 wherein the temperature of the first stage reaction mixture does not exceed 60° C. at least until the ratio of the number of equivalents of hydrogen reacted with β-farnesene, respectively, is exceeds 0.2:1.

Embodiment 99. The process of any of Embodiments 48-98 wherein the temperature of the first stage reaction mixture does not exceed 80° C. at least until the ratio of the number of equivalents of hydrogen reacted with β-farnesene, respectively, exceeds 0.9:1.

Embodiment 100. The process of any of Embodiments 48-99 wherein the temperature of the first stage reaction mixture does not exceed 100° C. at least until the ratio of the number of equivalents of hydrogen reacted with β-farnesene, respectively, exceeds 0.8:1.

Embodiment 101. The process of any of Embodiments 48-100 wherein the temperature of the first stage reaction mixture does not exceed 100° C. at least until the ratio of the number of equivalents of hydrogen reacted with β-farnesene, respectively, exceeds 0.8:1.

Embodiment 102. The process of any of Embodiments 48-101 wherein the temperature of the first stage reaction mixture does not exceed 120° C.

Embodiment 103. The process of any of Embodiments 48-102 wherein the temperature of the first stage reaction mixture does not exceed 160° C.

Embodiment 104. The process of any of Embodiments 48-103 wherein in the first stage the β-farnesene is reacted with at least about 0.9 equivalents of hydrogen per equivalent of β-farnesene.

Embodiment 105. The process of any of Embodiments 48-103 wherein in the first stage the β-farnesene is reacted with at least about 1 equivalents of hydrogen per equivalent of β-farnesene.

Embodiment 106. The process of any of Embodiments 48-103 wherein in the first stage the β-farnesene is reacted with at least about 1.1 equivalents of hydrogen per equivalent of β-farnesene.

Embodiment 107. The process of any of Embodiments 48-103 wherein in the first stage the β-farnesene is reacted with at least about 1.2 equivalents of hydrogen per equivalent of β-farnesene.

Embodiment 108. The process of any of Embodiments 48-107 wherein in the first stage the β-farnesene is reacted with less than 2 equivalents of hydrogen per equivalent of β-farnesene.

Embodiment 109. The process of any of Embodiments 48-107 wherein in the first stage the β-farnesene is reacted with less than 1.75 equivalents of hydrogen per equivalent of β-farnesene.

Embodiment 110. The process of any of Embodiments 48-107 wherein in the first stage the β-farnesene is reacted with less than 1.5 equivalents of hydrogen per equivalent of β-farnesene.

Embodiment 111. The process of any of Embodiments 48-107 wherein in the first stage the β-farnesene is reacted with less than 1.4 equivalents of hydrogen per equivalent of β-farnesene.

Embodiment 112. The process of any of Embodiments 48-107 wherein in the first stage the β-farnesene is reacted with less than 1.3 equivalents of hydrogen per equivalent of β-farnesene.

Embodiment 113. The process of any of Embodiments 48-107 wherein in the first stage the β-farnesene is reacted with less than 1.25 equivalents of hydrogen per equivalent of β-farnesene.

Embodiment 114. The process of any of Embodiments 48-113 wherein in the two stages, the β-farnesene is reacted with up to about 4 equivalents of hydrogen per equivalent of β-farnesene.

Embodiment 115. The process of any of Embodiments 48-113 wherein in the two stages, the β-farnesene is reacted with up to about 3.75 equivalents of hydrogen per equivalent of β-farnesene.

Embodiment 116. The process of any of Embodiments 48-113 wherein in the two stages, the β-farnesene is reacted with up to about 3.5 equivalents of hydrogen per equivalent of β-farnesene.

Embodiment 117. The process of any of Embodiments 48-113 wherein in the two stages, the β-farnesene is reacted with up to about 3.4 equivalents of hydrogen per equivalent of β-farnesene.

Embodiment 118. The process of any of Embodiments 48-113 wherein in the two stages, the β-farnesene is reacted with up to about 3.3 equivalents of hydrogen per equivalent of β-farnesene.

Embodiment 119. The process of any of Embodiments 48-113 wherein in the two stages, the β-farnesene is reacted with up to about 3.2 equivalents of hydrogen per equivalent of β-farnesene.

Embodiment 120. The process of any of Embodiments 48-113 wherein in the two stages, the β-farnesene is reacted with up to about 3.1 equivalents of hydrogen per equivalent of β-farnesene.

Embodiment 121. The process of any of Embodiments 48-120 wherein the first and second stage catalysts are independently selected from the group consisting of palladium, platinum, nickel, copper, copper-chromium, rhodium, ruthenium, silver and molybdenum catalysts.

Embodiment 122. The process of any of Embodiments 48-120 wherein the first and second stage catalysts are independently selected from the group consisting of palladium, platinum, and nickel catalysts.

Embodiment 123. The process of Embodiment 121 or 122 wherein the first and second stage catalysts are supported on a support selected from the group consisting of alumina, carbon, titanium, silicate, silica, titania, zirconia and alumina-silica.

Embodiment 124. The process of any of Embodiments 48 to 123 wherein the β-farnesene is produced by a microorganism.

Embodiment 125. The process of any of Embodiments 48-124 wherein the β-farnesene incorporates carbon from a renewable carbon source.

Embodiment 126. The process of any of Embodiments 48-124 wherein the β-farnesene comprises renewable carbon as determined in accordance with ASTM D6866-11.

Embodiment 127. The process of any of Embodiments 48-126 wherein first stage reaction mixture comprises a diluent.

Embodiment 128. The process of any of Embodiments 48-127 wherein first stage reaction product is recycled by removing it from a first, first stage reaction vessel in which the first stage reaction is being carried out and introducing it to a second, first stage reaction vessel in which the first stage reaction is being carried out.

Embodiment 129. The process of Embodiment 128 wherein the first and second, first stage reaction vessels are the same reaction vessel.

Embodiment 130. The process of Embodiment 128 wherein the first and second, first stage reaction vessels are different reaction vessels.

Embodiment 131. The process of any of Embodiments 128 to 130 wherein the first stage recycle rate is 100% to 900% of the rate of introduction of β-farnesene to the first stage.

Embodiment 132. The process of any of Embodiments 48-131 wherein the second stage reaction product is recycled by removing it from a first, second stage reaction vessel in which the second stage reaction is being carried out and introducing it to a second, second first stage reaction vessel in which the second stage reaction is being carried out.

Embodiment 133. The process of Embodiment 132 wherein the first and second, second stage reaction vessels are the same reaction vessel.

Embodiment 134. The process of Embodiment 132 wherein the first and second, second stage reaction vessels are different reaction vessels.

Embodiment 135. The process of any of Embodiments 132 to 134 wherein the second stage recycle rate is 100% to 900% of the rate of introduction of β-farnesene to the first stage.

Embodiment 136. The process of any of Embodiments 48 to 135 wherein the first stage and the second stage are independently carried out in continuous flow reactors operated adiabatically, isothermally or a combination thereof.

Embodiment 137. The process of Embodiment 136 wherein the first stage is carried out in the liquid phase in one or more reaction vessels.

Embodiment 138. The process of Embodiments 136 and 137 wherein the second stage is carried out in the vapor phase in one or more reaction vessels.

Embodiment 139. The process of any of Embodiments 48 to 138 wherein the first stage is carried out, at least in part, in one or more continuous stirred tank reactor.

Embodiment 140. The process of any of Embodiments 48 to 139 wherein the first stage reaction catalyst is recycled to the reactor in which the first stage reaction is carried out.

Embodiment 141. The process of any of Embodiments 48 to 139 wherein the first stage reaction catalyst is recycled to the reactor in which the second stage reaction is carried out.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Examples 1-5

### Preparation of Partially Hydrogenated Farnesene in a Continuous Fixed Bed Reactor System

β-farnesene was pre-treated with alumina, then slurried with palladium catalyst, and then pumped into a one gallon batch reactor. The reactor was vented and purged three times with 50 psig of nitrogen. Hydrogen was flowed into the reactor at 5 slpm (up to max pressure of 100 psig). Agitation was started while heating the reactor wall to 60° C. The reaction temperature was allowed to rise to 80° C. and then cooling water was used to hold the temperature at 80° C. until 1.2 equivalents of hydrogen were added. The temperature was then allowed to increase to 120° C. Cumulative hydrogen uptake was monitored using a hydrogen mass flow meter, totalizer and refractive index. After 1.5 equivalents of hydrogen were added, the reactor was heated to the second stage temperature of 260° C. After 3 molar equivalents of H₂ were consumed, hydrogen flow was stopped and excess H₂ was vented. Agitation was reduced and the reactor cooled to at least 50° C. The reactor was purged twice with 20 psig of nitrogen during the cool down. The product was then separated from the catalyst by diatomaceous earth filtration and the reaction product analyzed by GC-FID yielding the following results:

| | | Stage 1 | | Stage 2 | Stage 2 | | | Hexahydro- | Tetrahydro- | Dihydro- | |
|---|---|---|---|---|---|---|---|---|---|---|---|
| | | Pressure | Stage 1 | Pressure | Temp | Extent of | Farnesane | farnesene | farnesene | farnesene | Farnesene |
| Example # | Catalyst | (psig) | Temp (° C.) | (psig) | (° C.) | Hydrogenation | (area %) | (area %) | (area %) | (area %) | (area %) |
| 1 | 0.3 wt % | 100 | 80 | 10 | 220 | 2.99 | 4.1 | 90.6 | 5.2 | 0.1 | 0.0 |
| | Pd/Al₂O₃ | | | | | | | | | | |
| 2 | 0.3 wt % | 100 | 80 | 30 | 260 | 3.01 | 7.0 | 87.0 | 5.7 | 0.1 | 0.0 |
| | Pd/Al₂O₃ | | | | | | | | | | |
| 3 | 0.6 wt % | 100 | 80 | 30 | 260 | 3.04 | 8.2 | 87.5 | 4.1 | 0.1 | 0.0 |
| | Pd/Al₂O₃ | | | | | | | | | | |
| 4 | 0.5 wt % | 100 | 80 | 30 | 260 | 3.00 | 7.5 | 85.2 | 7.1 | 0.1 | 0.0 |
| | Pd/C | | | | | | | | | | |
| | Support #1 | | | | | | | | | | |
| 5 | 0.5 wt % | 100 | 80 | 30 | 260 | 3.02 | 7.5 | 87.4 | 5.0 | 0.1 | 0.0 |
| | Pd/C | | | | | | | | | | |
| | Support #2 | | | | | | | | | | |

### Examples 6-13

### Effect of Temperature and Pressure on Second Stage Selectivity

Examples 6-13 were carried out with the same procedure described for examples 1-5 using 0.3% Pd/Al₂O₃ with further variation on temperature and pressure in the second stage. Examples 6-8 show that at lower temperatures, low pressures are needed to reach selectivity to >85% hexahydro-β-farnesene, but selectivity can be regained at higher temperatures even at higher second stage pressures.

| | Stage 2 | Stage 2A | Stage 2B | | Hexahydro- | Tetrahydro- | |
|---|---|---|---|---|---|---|---|
| | Temp | Pressure | Pressure | Ave # H | farnesene | farnesene | Farnesane |
| Example # | (° C.) | (psig) | (psig) | Bonds | (area %) | (area %) | (area %) |
| 6 | 220 | 10 | 5 | 2.97 | 90.59 | 3.52 | 4.10 |
| 7 | 220 | 10 | 10 | 3.00 | 89.38 | 3.91 | 5.79 |
| 8 | 220 | 15 | 15 | 3.02 | 85.54 | 4.33 | 8.79 |
| 9 | 230 | 20 | 15 | 3.01 | 88.15 | 4.28 | 6.70 |
| 10 | 200 | 30 | 20 | 3.00 | 76.33 | 11.19 | 11.75 |
| 11 | 240 | 30 | 20 | 2.92 | 82.21 | 12.05 | 4.99 |
| 12 | 260 | 30 | 20 | 3.02 | 89.28 | 3.31 | 6.59 |
| 13 | 260 | 40 | 40 | 3.02 | 86.20 | 5.54 | 8.09 |

### Example 14

### Preparation of Partially Hydrogenated Farnesene in a 7000 Gallon Batch Reactor

The process of the present disclosure was scaled up to a 7000 gallon batch reactor. Activated alumina spheres were used to treat β-Farnesene in a stainless steel 3940 gallons column. The hydrogenation reactor was purged with 30 psig of nitrogen twice. The treated β-farnesene was transferred to the reactor and the agitator was turned on after about half of the β-farnesene was charged. The reactor was heated to 60° C., catalyst was introduced to the reactor, and a vacuum to 50 mmHg or less was pulled in the reactor. Hydrogen was fed while the reactor was heated to 80° C. by reaction exotherm. First stage reaction temperature was maintained until 1.5 equivalents of hydrogen were added as a more conservative approach to minimize formation of higher molecular weight farnesene derivatives, due to greater mixing and heat removal challenges in a larger reactor. Analysis of the first stage reaction product (by GC analysis) show <1% Farnesene and <1% of Farnesane. After 1.5 equivalents of hydrogen were added as measured by the totalizer, the heat of reaction from hydrogenation was used to heat the reactor to second stage conditions of 260-275° C. for selectivity to hexahydro β-farnesene while minimizing the formation of farnesane.

### Example 15

### Preparation of Partially Hydrogenated Farnesene in One or More Batch Slurry Reactors with Catalyst Recycle

The multi-stage process of the present disclosure was carried out in a batch slurry reactor using a 0.3% Pd catalyst supported on alumina powder with an initial concentration of 18 ppm of Pd metal. After 3 molar equivalents of H₂ were consumed, hydrogen flow was stopped and excess hydrogen was vented. The reactor was purged and cooled. The product was discharged and separated from the used Pd catalyst by gravimetric separation. The used catalyst was returned to the reactor with fresh feed and additional new catalyst containing 6 ppm of fresh Pd. The multi-stage reaction was carried out 4 (four) additional times, returning the used catalyst with 6 ppm of fresh catalyst on each subsequent run. The resulting product from each run was analyzed by GC-FID giving the following results:

| | | | | Hexahydro- | Dihydro- | |
|---|---|---|---|---|---|---|
| Reaction | Fresh Pd | Ave # H | Farnesane | farnesene | farnesene | Refractive |
| Cycle # | (ppm) | Bonds | (area %) | (area %) | (area %) | Index |
| R0 | 18 | 2.96 | 4.16 | 87.31 | 7.93 | 1.4431 |
| R1 | 6 | 2.94 | 4.12 | 85.9 | 9.48 | 1.4435 |
| R2 | 6 | 2.99 | 6.74 | 85.69 | 7.17 | 1.443 |
| R3 | 6 | 2.98 | 6.93 | 84.5 | 8.17 | 1.443 |
| R4 | 6 | 3 | 7.08 | 85.57 | 7.01 | 1.443 |
| R5 | 6 | 3.01 | 8.15 | 85.04 | 6.55 | 1.443 |

| | # of | wt % of | | wt % of | wt % of | |
|---|---|---|---|---|---|---|
| | Hydrogenated | Hexahydro- | wt % of | Tetrahydro- | Dihydro- | wt % of β- |
| Example # | Double Bonds | farnesene | Farnesane | farnesene | farnesene | farnesene |
| 14 Stage | 1.49 | 7.68 | 0.09 | 33.57 | 58.07 | 0.00 |
| 1 Product | | | | | | |
| 14 Stage | 3.01 | 89.57 | 5.96 | 3.86 | 0.53 | 0.00 |
| 2 Product | | | | | | |

### Example 15-17

### Preparation of First Stage Partial Hydrogenation of Farnesene in a Continuous Fixed Bed Reactor System

Distilled β-farnesene was filtered through 2″ ID×48″ Height column filled with Selexsorb CDX. Johnson Matthey, Pricat PD 309/45 0.5% Pd/Alumina trilobes, 2.5 mm were loaded to a 2″ID×48″ height adiabatic reactor. Catalyst was added in four zones with a liquid distributor on top of each catalyst bed. After catalyst activation, the bed was cooled to about 60° C. then the reactor was pressurized to 30 psig. Excess hydrogen was added to prevent starving the catalyst. Hydrogen flow exiting the bed was maintained at 0.5 slpm or higher. Selective hydrogenation was run continuously for 100 hours. Analysis of the composition of the product by GC-MS & GC-FID showed that 1.4-1.6 equivalents of hydrogen had been added with the following distribution of species:

| | | Stage 1 | Stage 1 Inlet | | | | Hexahydro- | Tetrahydro- | | |
|---|---|---|---|---|---|---|---|---|---|---|
| | | Pressure | Temperature | | Extent of | Farnesane | farnesene | farnesene | Dihydrofarnesene | Farnesene |
| Example | Catalyst | (psig) | (° C.) | LHSV | Hydrogenation | (area %) | (area %) | (area %) | (area %) | (area %) |
| 15 | PRICAT PD | 30 | 55 | 0.6 | 1.6 | 0.5 | 12.5 | 33.8 | 52.5 | 0.5 |
| 16 | PRICAT PD | 50 | 55 | 1.0 | 1.5 | 0.5 | 10.3 | 31.7 | 56.0 | 0.7 |
| 17 | HTC NI | 70 | 60 | 0.9 | 1.4 | 1.0 | 5.9 | 26.5 | 65.6 | 1.0 |

What is claimed is:

1. An olefinic product comprising partially hydrogenated β-farnesene, the olefinic product having (i) a mass fraction of farnesane that is no more than 1 wt % of the combined amount of β-farnesene, partially hydrogenated β-farnesene and farnesane in the olefinic product, (ii) a mass fraction of β-farnesene that is no more than 5 wt % of the combined amount of β-farnesene, partially hydrogenated β-farnesene and farnesane in the olefinic product, and (iii) a mass fraction of β-farnesene derivatives having a molecular weight greater than farnesane that is no more than 2.5 wt % of the combined amount of β-farnesene, partially hydrogenated β-farnesene, farnesane and β-farnesene derivatives having a molecular weight greater than farnesane.

2. The olefinic product of claim 1 wherein the mass fraction of dihydro-β-farnesene is at least 85 wt % of the combined amount of β-farnesene, partially hydrogenated β-farnesene, and farnesane in the olefinic product.

3. The olefinic product of claim 1 wherein the mass fraction of dihydro-β-farnesene is at least 90 wt % of the combined amount of β-farnesene, partially hydrogenated β-farnesene, and farnesane in the olefinic product.

4. The olefinic product of claim 1 wherein the mass fraction of dihydro-β-farnesene is at least 92 wt % of the combined amount of β-farnesene, partially hydrogenated β-farnesene, and farnesane in the olefinic product.

5. The olefinic product of claim 1 wherein the mass fraction of dihydro-β-farnesene is at least 94 wt % of the combined amount of β-farnesene, partially hydrogenated β-farnesene, and farnesane in the olefinic product.

6. The olefinic product of claim 1 wherein the mass fraction of dihydro-β-farnesene is at least 96 wt % of the combined amount of β-farnesene, partially hydrogenated β-farnesene, and farnesane in the olefinic product.

7. The olefinic product of any preceding claim wherein the mass fraction of partially hydrogenated β-farnesene is at least 95 wt % of the combined amount of β-farnesene, partially hydrogenated β-farnesene, and farnesane in the olefinic product.

8. The olefinic product of any preceding claim wherein the mass fraction of partially hydrogenated β-farnesene is at least 97 wt % of the combined amount of β-farnesene, partially hydrogenated β-farnesene, and farnesane in the olefinic product.

9. The olefinic product of any preceding claim wherein the mass fraction of partially hydrogenated β-farnesene is at least 98 wt % of the combined amount of β-farnesene, partially hydrogenated β-farnesene, and farnesane in the olefinic product.

10. The olefinic product of any preceding claim wherein the mass fraction of partially hydrogenated β-farnesene is at least 99 wt % of the combined amount of β-farnesene, partially hydrogenated β-farnesene, and farnesane in the olefinic product.

11. The olefinic product of any preceding claim wherein the mass fraction of β-farnesene in the olefinic product is no more than 4 wt % of the combined amount of β-farnesene, partially hydrogenated β-farnesene, and farnesane in the olefinic product.

12. The olefinic product of any preceding claim wherein the mass fraction of β-farnesene in the olefinic product is no more than 3 wt % of the combined amount of β-farnesene, partially hydrogenated β-farnesene, and farnesane in the olefinic product.

13. The olefinic product of any preceding claim wherein the mass fraction of (β-farnesene in the olefinic product is no more than 2 wt % of the combined amount of (β-farnesene, partially hydrogenated β-farnesene, and farnesane in the olefinic product.

14. The olefinic product of any preceding claim wherein the mass fraction of (β-farnesene in the olefinic product is no more than 1 wt % of the combined amount of (β-farnesene, partially hydrogenated β-farnesene, and farnesane in the olefinic product.

15. The olefinic product of any preceding claim wherein the mass fraction of β-farnesene in the olefinic product is no more than 0.5 wt % of the combined amount of β-farnesene, partially hydrogenated β-farnesene, and farnesane in the olefinic product.

16. The olefinic product of any preceding claim wherein the mass fraction of β-farnesene in the olefinic product is no more than 0.25 wt % of the combined amount of β-farnesene, partially hydrogenated β-farnesene, and farnesane in the olefinic product.

17. The olefinic product of any preceding claim wherein the mass fraction of β-farnesene in the olefinic product is no more than 0.1 wt % of the combined amount of β-farnesene, partially hydrogenated β-farnesene, and farnesane in the olefinic product.

18. The olefinic product of any preceding claim wherein β-farnesene is undetectable in the olefinic product.

19. The olefinic product of any preceding claim wherein the mass fraction of farnesane in the olefinic product is no more than 0.75 wt % of the combined amount of β-farnesene, partially hydrogenated β-farnesene, and farnesane in the olefinic product.

20. The olefinic product of any preceding claim wherein the mass fraction of farnesane in the olefinic product is no more than 0.5 wt % of the combined amount of β-farnesene, partially hydrogenated β-farnesene, and farnesane in the olefinic product.

21. The olefinic product of any preceding claim wherein the mass fraction of farnesane in the olefinic product is no more than 0.25 wt % of the combined amount of β-farnesene, partially hydrogenated β-farnesene, and farnesane in the olefinic product.

22. The olefinic product of any preceding claim wherein the mass fraction of farnesane in the olefinic product is no more than 0.1 wt % of the combined amount of β-farnesene, partially hydrogenated β-farnesene, and farnesane in the olefinic product.

23. The olefinic product of any preceding claim wherein the mass fraction of farnesane in the olefinic product is no more than 0.05 wt % of the combined amount of β-farnesene, partially hydrogenated β-farnesene, and farnesane in the olefinic product.

24. The olefinic product of any preceding claim wherein farnesane is undetectable in the olefinic product.

25. An olefinic product comprising hexahydro-β-farnesene, the olefinic product having (i) a mass fraction of farnesane that is no more than 7 wt % relative to the combined amount of β-farnesene, partially hydrogenated β-farnesene and farnesane in the second stage reaction product, (ii) a mass fraction of hexahydro-β-farnesene that is at least 85 wt % of the combined amount of β-farnesene, partially hydrogenated β-farnesene and farnesane in the second stage reaction product, and (iii) a mass fraction of β-farnesene derivatives having a molecular weight greater than farnesane that is no more than 2.5 wt % of the combined amount of β-farnesene, partially hydrogenated β-farnesene, farnesane and β-farnesene derivatives having a molecular weight greater than farnesane in the second stage reaction product.

26. The olefinic product of claim 25 wherein the mass fraction of β-farnesene in the olefinic product is no more than 0.05 wt % of the combined amount of β-farnesene, partially hydrogenated β-farnesene, and farnesane in the olefinic product.

27. The olefinic product of claim 25 wherein β-farnesene is undetectable in the olefinic product.

28. The olefinic product of any of claims 25 to 27 wherein the mass fraction of hexahydro-β-farnesene is at least 87 wt % of the combined amount of β-farnesene, partially hydrogenated β-farnesene, and farnesane in the olefinic product.

29. The olefinic product of any of claims 25 to 27 wherein the mass fraction of hexahydro-β-farnesene is at least 88 wt % of the combined amount of β-farnesene, partially hydrogenated β-farnesene, and farnesane in the olefinic product.

30. The olefinic product of any of claims 25 to 27 wherein the mass fraction of hexahydro-β-farnesene is at least 89 wt % of the combined amount of β-farnesene, partially hydrogenated β-farnesene, and farnesane in the olefinic product.

31. The olefinic product of any of claims 25 to 27 wherein the mass fraction of hexahydro-β-farnesene is at least 90 wt % of the combined amount of β-farnesene, partially hydrogenated β-farnesene, and farnesane in the olefinic product.

32. The olefinic product of any of claims 25 to 27 wherein the mass fraction of hexahydro-β-farnesene is at least 91 wt % of the combined amount of β-farnesene, partially hydrogenated β-farnesene, and farnesane in the olefinic product.

33. The olefinic product of any of claims 25 to 27 wherein the mass fraction of hexahydro-β-farnesene is at least 92 wt % of the combined amount of β-farnesene, partially hydrogenated β-farnesene, and farnesane in the olefinic product.

34. The olefinic product of any of claims 25 to 27 wherein the mass fraction of hexahydro-β-farnesene is at least 93 wt % of the combined amount of β-farnesene, partially hydrogenated β-farnesene, and farnesane in the olefinic product.

35. The olefinic product of any of claims 25 to 34 wherein the mass fraction of farnesane in the olefinic product is no more than 6 wt % of the combined amount of β-farnesene, partially hydrogenated β-farnesene, and farnesane in the olefinic product.

36. The olefinic product of any of claims 25 to 34 wherein the mass fraction of farnesane in the olefinic product is no more than 5 wt % of the combined amount of β-farnesene, partially hydrogenated β-farnesene, and farnesane in the olefinic product.

37. The olefinic product of any of claims 25 to 34 wherein the mass fraction of farnesane in the olefinic product is no more than 4 wt % of the combined amount of β-farnesene, partially hydrogenated β-farnesene, and farnesane in the olefinic product.

38. The olefinic product of any of claims 25 to 34 wherein the mass fraction of farnesane in the olefinic product is no more than 3 wt % of the combined amount of β-farnesene, partially hydrogenated β-farnesene, and farnesane in the olefinic product.

39. The olefinic product of any of claims 25 to 34 wherein the mass fraction of farnesane in the olefinic product is no more than 2 wt % of the combined amount of β-farnesene, partially hydrogenated β-farnesene, and farnesane in the olefinic product.

40. The olefinic product of any of claims 25 to 34 wherein the mass fraction of farnesane in the olefinic product is no more than 1 wt % of the combined amount of β-farnesene, partially hydrogenated β-farnesene, and farnesane in the olefinic product.

41. The olefinic product of any preceding claim wherein the mass fraction of β-farnesene derivatives having a molecular weight greater than farnesane is no more than 2 wt % of the combined amount of β-farnesene, partially hydrogenated β-farnesene, farnesane, and β-farnesene derivatives having a molecular weight greater than farnesane in the olefinic product.

42. The olefinic product of any preceding claim wherein the mass fraction of β-farnesene derivatives having a molecular weight greater than farnesane is no more than 1.5 wt % of the combined amount of β-farnesene, partially hydrogenated β-farnesene, farnesane, and β-farnesene derivatives having a molecular weight greater than farnesane in the olefinic product.

43. The olefinic product of any preceding claim wherein the mass fraction of β-farnesene derivatives having a molecular weight greater than farnesane is no more than 1 wt % of the combined amount of β-farnesene, partially hydrogenated β-farnesene, farnesane, and β-farnesene derivatives having a molecular weight greater than farnesane in the olefinic product.

44. The olefinic product of any preceding claim wherein the mass fraction of β-farnesene derivatives having a molecular weight greater than farnesane is no more than 0.5 wt % of the combined amount of β-farnesene, partially hydrogenated β-farnesene, farnesane, and β-farnesene derivatives having a molecular weight greater than farnesane in the olefinic product.

45. The olefinic product of any preceding claim wherein the mass fraction of β-farnesene derivatives having a molecular weight greater than farnesane is no more than 0.25 wt % of the combined amount of β-farnesene, partially hydrogenated β-farnesene, farnesane, and β-farnesene derivatives having a molecular weight greater than farnesane in the olefinic product.

46. The olefinic product of any preceding claim wherein the mass fraction of β-farnesene derivatives having a molecular weight greater than farnesane is no more than 0.1 wt % of the combined amount of β-farnesene, partially hydrogenated β-farnesene, farnesane, and β-farnesene derivatives having a molecular weight greater than farnesane in the olefinic product.

47. The olefinic product of any preceding claim wherein β-farnesene derivatives having a molecular weight greater than farnesane are undetectable in the olefinic product.

48. A process for hydrogenating β-farnesene to form an olefinic composition, the process comprising:
(a) in a first stage, reacting the β-farnesene with hydrogen in the presence of a first stage catalyst in a first stage reaction mixture while controlling the rate of hydrogenation of the β-farnesene relative to the rate of formation of β-farnesene derivatives having a molecular weight greater than farnesane to produce a first stage reaction product wherein (i) the mass fraction of farnesane in the first stage reaction product is no more than 1 wt % of the combined amount of β-farnesene, partially hydrogenated β-farnesene and farnesane in the first stage reaction product, (ii) the mass fraction of β-farnesene in the first stage reaction product is no more than 5 wt % of the combined amount of β-farnesene, partially hydrogenated β-farnesene and farnesane in the first stage reaction product, and (iii) the mass fraction of β-farnesene derivatives having a molecular weight greater than farnesane in the first stage reaction product is no more than 2.5 wt % of the combined amount of β-farnesene, partially hydrogenated β-farnesene, farnesane and β-farnesene derivatives having a molecular weight greater than farnesane in the first stage reaction product, and
(b) in a second stage, reacting the first stage reaction product with hydrogen in the presence of a second stage catalyst to produce a second stage reaction product, wherein (i) the mass fraction of farnesane in the second stage reaction product is no more than 7 wt % of the combined amount of β-farnesene, partially hydrogenated β-farnesene and farnesane in the second stage reaction product, (ii) the mass fraction of partially hexahydro-β-farnesene in the second stage reaction product is at least 85 wt % of the combined amount of β-farnesene, partially hydrogenated β-farnesene and farnesane in the second stage reaction product, and (iii) the mass fraction of β-farnesene derivatives having a molecular weight greater than farnesane in the second stage reaction product is no more than 2.5 wt % of the combined amount of β-farnesene, partially hydrogenated β-farnesene, farnesane and β-farnesene derivatives having a molecular weight greater than farnesane in the second stage reaction product.

49. The process of claim 48 wherein the mass fraction of dihydro-β-farnesene is at least 85 wt % of the combined amount of β-farnesene, partially hydrogenated β-farnesene, and farnesane in the first stage reaction product.

50. The process of claim 48 wherein the mass fraction of dihydro-β-farnesene is at least 90 wt % of the combined amount of β-farnesene, partially hydrogenated β-farnesene, and farnesane in the first stage reaction product.

51. The process of claim 48 wherein the mass fraction of dihydro-β-farnesene is at least 92 wt % of the combined amount of β-farnesene, partially hydrogenated (β-farnesene, and farnesane in the first stage reaction product.

52. The process of claim 48 wherein the mass fraction of dihydro-β-farnesene is at least 94 wt % of the combined amount of β-farnesene, partially hydrogenated (β-farnesene, and farnesane in the first stage reaction product.

53. The process of claim 48 wherein the mass fraction of dihydro-β-farnesene is at least 96 wt % of the combined amount of β-farnesene, partially hydrogenated β-farnesene, and farnesane in the first stage reaction product.

54. The process of any of claims 48 to 53 wherein the mass fraction of partially hydrogenated β-farnesene is at least 96 wt % of the combined amount of β-farnesene, partially hydrogenated β-farnesene, and farnesane in the first stage reaction product.

55. The process of any of claims 48 to 53 wherein the mass fraction of partially hydrogenated β-farnesene is at least 97 wt % of the combined amount of β-farnesene, partially hydrogenated β-farnesene, and farnesane in the first stage reaction product.

56. The process of any of claims 48 to 53 wherein the mass fraction of partially hydrogenated β-farnesene is at least 98 wt % of the combined amount of β-farnesene, partially hydrogenated β-farnesene, and farnesane in the first stage reaction product.

57. The process of any of claims 48 to 53 wherein the mass fraction of partially hydrogenated β-farnesene is at least 99 wt % of the combined amount of β-farnesene, partially hydrogenated β-farnesene, and farnesane in the first stage reaction product.

58. The process of any of claims 48 to 57 wherein the mass fraction of partially hydrogenated β-farnesene is no more than 4 wt % of the combined amount of β-farnesene, partially hydrogenated β-farnesene, and farnesane in the first stage reaction product.

59. The process of any of claims 48 to 57 wherein the mass fraction of partially hydrogenated β-farnesene is no more than 3 wt % of the combined amount of β-farnesene, partially hydrogenated β-farnesene, and farnesane in the first stage reaction product.

60. The process of any of claims 48 to 57 wherein the mass fraction of partially hydrogenated β-farnesene is no more than 2 wt % of the combined amount of β-farnesene, partially hydrogenated β-farnesene, and farnesane in the first stage reaction product.

61. The process of any of claims 48 to 57 wherein the mass fraction of partially hydrogenated β-farnesene is no more than 1 wt % of the combined amount of β-farnesene, partially hydrogenated β-farnesene, and farnesane in the first stage reaction product.

62. The process of any of claims 48 to 57 wherein the mass fraction of partially hydrogenated β-farnesene is no more than 0.5 wt % of the combined amount of β-farnesene, partially hydrogenated β-farnesene, and farnesane in the first stage reaction product.

63. The process of any of claims 48 to 57 wherein the mass fraction of partially hydrogenated β-farnesene is no more than 0.25 wt % of the combined amount of β-farnesene, partially hydrogenated β-farnesene, and farnesane in the first stage reaction product.

64. The process of any of claims 48 to 57 wherein the mass fraction of partially hydrogenated β-farnesene is no more than 0.1 wt % of the combined amount of β-farnesene, partially hydrogenated β-farnesene, and farnesane in the first stage reaction product.

65. The process of any of claims 48 to 57 wherein β-farnesene is undetectable in the first stage reaction product.

66. The process of any of claims 48 to 65 wherein the mass fraction of farnesane in the first stage reaction product is no more than 0.75 wt % of the combined amount of β-farnesene, partially hydrogenated β-farnesene, and farnesane in the first stage reaction product.

67. The process of any of claims 48 to 65 wherein the mass fraction of farnesane in the first stage reaction product is no more than 0.5 wt % of the combined amount of β-farnesene, partially hydrogenated β-farnesene, and farnesane in the first stage reaction product.

68. The process of any of claims 48 to 65 wherein the mass fraction of farnesane in the first stage reaction product is no more than 0.25 wt % of the combined amount of β-farnesene, partially hydrogenated β-farnesene, and farnesane in the first stage reaction product.

69. The process of any of claims 48 to 65 wherein the mass fraction of farnesane in the first stage reaction product is no more than 0.1 wt % of the combined amount of β-farnesene, partially hydrogenated β-farnesene, and farnesane in the first stage reaction product.

70. The process of any of claims 48 to 65 wherein the mass fraction of farnesane in the first stage reaction product is no more than 0.05 wt % of the combined amount of β-farnesene, partially hydrogenated β-farnesene, and farnesane in the first stage reaction product.

71. The process of any of claims 48 to 70 wherein the first stage reaction product is treated to remove β-farnesene derivatives having a molecular weight greater than farnesane before the first stage reaction product is reacted with hydrogen in the presence of a second stage catalyst to produce a second stage reaction product.

72. The process of any of claims 48 to 70 wherein the first stage reaction product is treated in a thin film evaporator, a wiped film evaporator, or a distillation column to remove β-farnesene derivatives having a molecular weight greater than farnesane before the first stage reaction product is reacted with hydrogen in the presence of a second stage catalyst to produce a second stage reaction product.

73. The process of any of claims 48 to 72 wherein the mass fraction of β-farnesene in the second stage reaction product is no more than 0.05 wt % of the combined amount of β-farnesene, partially hydrogenated β-farnesene, and farnesane in the second stage reaction product.

74. The process of any of claims 48 to 72 wherein β-farnesene is undetectable in the second stage reaction product.

75. The process of any of claims 48 to 74 wherein the mass fraction of hexahydro-β-farnesene is at least 87 wt % of the combined amount of β-farnesene, partially hydrogenated β-farnesene, and farnesane in the second stage reaction product.

76. The process of any of claims 48 to 74 wherein the mass fraction of hexahydro-β-farnesene is at least 88 wt % of the combined amount of β-farnesene, partially hydrogenated β-farnesene, and farnesane in the second stage reaction product.

77. The process of any of claims 48 to 74 wherein the mass fraction of hexahydro-β-farnesene is at least 89 wt % of the combined amount of β-farnesene, partially hydrogenated β-farnesene, and farnesane in the second stage reaction product.

78. The process of any of claims 48 to 74 wherein the mass fraction of hexahydro-β-farnesene is at least 90 wt % of the combined amount of β-farnesene, partially hydrogenated β-farnesene, and farnesane in the second stage reaction product.

79. The process of any of claims 48 to 74 wherein the mass fraction of hexahydro-β-farnesene is at least 91 wt % of the combined amount of β-farnesene, partially hydrogenated β-farnesene, and farnesane in the second stage reaction product.

80. The process of any of claims 48 to 74 wherein the mass fraction of hexahydro-β-farnesene is at least 92 wt % of the combined amount of β-farnesene, partially hydrogenated β-farnesene, and farnesane in the second stage reaction product.

81. The process of any of claims 48 to 74 wherein the mass fraction of hexahydro-β-farnesene is at least 93 wt % of the combined amount of β-farnesene, partially hydrogenated β-farnesene, and farnesane in the second stage reaction product.

82. The process of any of claims 48 to 81 wherein the mass fraction of farnesane in the second stage reaction product is no more than 6 wt % of the combined amount of β-farnesene, partially hydrogenated β-farnesene, and farnesane in the second stage reaction product.

83. The process of any of claims 48 to 81 wherein the mass fraction of farnesane in the second stage reaction product is no more than 5 wt % of the combined amount of β-farnesene, partially hydrogenated β-farnesene, and farnesane in the second stage reaction product.

84. The process of any of claims 48 to 81 wherein the mass fraction of farnesane in the second stage reaction product is no more than 4 wt % of the combined amount of β-farnesene, partially hydrogenated β-farnesene, and farnesane in the second stage reaction product.

85. The process of any of claims 48 to 81 wherein the mass fraction of farnesane in the second stage reaction product is no more than 3 wt % of the combined amount of β-farnesene, partially hydrogenated β-farnesene, and farnesane in the second stage reaction product.

86. The process of any of claims 48 to 81 wherein the mass fraction of farnesane in the second stage reaction product is no more than 2 wt % of the combined amount of β-farnesene, partially hydrogenated β-farnesene, and farnesane in the second stage reaction product.

87. The process of any of claims 48 to 81 wherein the mass fraction of farnesane in the second stage reaction product is no more than 1 wt % of the combined amount of β-farnesene, partially hydrogenated β-farnesene, and farnesane in the second stage reaction product.

88. The process of any of claims 48 to 87 wherein the mass fraction of β-farnesene derivatives having a molecular weight greater than farnesane is no more than 2 wt % of the combined amount of β-farnesene, partially hydrogenated β-farnesene, farnesane, and β-farnesene derivatives having a molecular weight greater than farnesane in the first and second stage reaction products.

89. The process of any of claims 48 to 87 wherein the mass fraction of β-farnesene derivatives having a molecular weight greater than farnesane is no more than 1.5 wt % of the combined amount of β-farnesene, partially hydrogenated β-farnesene, farnesane, and β-farnesene derivatives having a molecular weight greater than farnesane in the first and second stage reaction products.

90. The process of any of claims 48 to 87 wherein the mass fraction of β-farnesene derivatives having a molecular weight greater than farnesane is no more than 1 wt % of the combined amount of β-farnesene, partially hydrogenated β-farnesene, farnesane, and β-farnesene derivatives having a molecular weight greater than farnesane in the first and second stage reaction products.

91. The process of any of claims 48 to 87 wherein the mass fraction of β-farnesene derivatives having a molecular weight greater than farnesane is no more than 0.5 wt % of the combined amount of β-farnesene, partially hydrogenated β-farnesene, farnesane, and β-farnesene derivatives having a molecular weight greater than farnesane in the first and second stage reaction products.

92. The process of any of claims 48 to 87 wherein the mass fraction of (β-farnesene derivatives having a molecular weight greater than farnesane is no more than 0.25 wt % of the combined amount of β-farnesene, partially hydrogenated β-farnesene, farnesane, and β-farnesene derivatives having a molecular weight greater than farnesane in the first and second stage reaction products.

93. The process of any of claims 48 to 87 wherein the mass fraction of β-farnesene derivatives having a molecular weight greater than farnesane is no more than 0.1 wt % of the combined amount of β-farnesene, partially hydrogenated β-farnesene, farnesane, and β-farnesene derivatives having a molecular weight greater than farnesane in the first and second stage reaction products.

94. The process of any of claims 48-93 wherein the temperature of the first stage reaction mixture is increased from a temperature at or near room temperature to a maximum temperature of about 120° C. as the amount of β-farnesene decreases and the amount of partially hydrogenated β-farnesene increases in the first stage reaction mixture.

95. The process of any of claims 48-93 wherein the temperature of the first stage reaction mixture is increased from a temperature at or near room temperature to a maximum temperature of about 100° C. as the amount of β-farnesene decreases and the amount of partially hydrogenated β-farnesene increases in the first stage reaction mixture.

96. The process of any of claims 48-93 wherein the temperature of the first stage reaction mixture is increased from a temperature at or near room temperature to a maximum temperature in the range of about 80 to 100° C. as the amount of β-farnesene decreases and the amount of partially hydrogenated β-farnesene increases in the first stage reaction mixture.

97. The process of any of claims 48-96 wherein the temperature of the first stage reaction mixture does not exceed 50° C. at least until the ratio of the number of equivalents of hydrogen reacted with β-farnesene, respectively, exceeds 0.1:1.

98. The process of any of claims 48-97 wherein the temperature of the first stage reaction mixture does not exceed 60° C. at least until the ratio of the number of equivalents of hydrogen reacted with β-farnesene, respectively, is exceeds 0.2:1.

99. The process of any of claims 48-98 wherein the temperature of the first stage reaction mixture does not exceed 80° C. at least until the ratio of the number of equivalents of hydrogen reacted with β-farnesene, respectively, exceeds 0.9:1.

100. The process of any of claims 48-99 wherein the temperature of the first stage reaction mixture does not exceed 100° C. at least until the ratio of the number of equivalents of hydrogen reacted with β-farnesene, respectively, exceeds 0.8:1.

101. The process of any of claims 48-100 wherein the temperature of the first stage reaction mixture does not exceed 100° C. at least until the ratio of the number of equivalents of hydrogen reacted with β-farnesene, respectively, exceeds 0.8:1.

102. The process of any of claims 48-101 wherein the temperature of the first stage reaction mixture does not exceed 120° C.

103. The process of any of claims 48-102 wherein the temperature of the first stage reaction mixture does not exceed 160° C.

104. The process of any of claims 48-103 wherein in the first stage the β-farnesene is reacted with at least about 0.9 equivalents of hydrogen per equivalent of β-farnesene.

105. The process of any of claims 48-103 wherein in the first stage the β-farnesene is reacted with at least about 1 equivalents of hydrogen per equivalent of β-farnesene.

106. The process of any of claims 48-103 wherein in the first stage the β-farnesene is reacted with at least about 1.1 equivalents of hydrogen per equivalent of β-farnesene.

107. The process of any of claims 48-103 wherein in the first stage the β-farnesene is reacted with at least about 1.2 equivalents of hydrogen per equivalent of β-farnesene.

108. The process of any of claims 48-107 wherein in the first stage the β-farnesene is reacted with less than 2 equivalents of hydrogen per equivalent of β-farnesene.

109. The process of any of claims 48-107 wherein in the first stage the β-farnesene is reacted with less than 1.75 equivalents of hydrogen per equivalent of β-farnesene.

110. The process of any of claims 48-107 wherein in the first stage the β-farnesene is reacted with less than 1.5 equivalents of hydrogen per equivalent of β-farnesene.

111. The process of any of claims 48-107 wherein in the first stage the β-farnesene is reacted with less than 1.4 equivalents of hydrogen per equivalent of β-farnesene.

112. The process of any of claims 48-107 wherein in the first stage the β-farnesene is reacted with less than 1.3 equivalents of hydrogen per equivalent of β-farnesene.

113. The process of any of claims 48-107 wherein in the first stage the β-farnesene is reacted with less than 1.25 equivalents of hydrogen per equivalent of β-farnesene.

114. The process of any of claims 48-113 wherein in the two stages, the (β-farnesene is reacted with up to about 4 equivalents of hydrogen per equivalent of (β-farnesene.

115. The process of any of claims 48-113 wherein in the two stages, the (β-farnesene is reacted with up to about 3.75 equivalents of hydrogen per equivalent of (β-farnesene.

116. The process of any of claims 48-113 wherein in the two stages, the β-farnesene is reacted with up to about 3.5 equivalents of hydrogen per equivalent of β-farnesene.

117. The process of any of claims 48-113 wherein in the two stages, the β-farnesene is reacted with up to about 3.4 equivalents of hydrogen per equivalent of β-farnesene.

118. The process of any of claims 48-113 wherein in the two stages, the β-farnesene is reacted with up to about 3.3 equivalents of hydrogen per equivalent of β-farnesene.

119. The process of any of claims 48-113 wherein in the two stages, the β-farnesene is reacted with up to about 3.2 equivalents of hydrogen per equivalent of β-farnesene.

120. The process of any of claims 48-113 wherein in the two stages, the β-farnesene is reacted with up to about 3.1 equivalents of hydrogen per equivalent of β-farnesene.

121. The process of any of claims 48-120 wherein the first and second stage catalysts are independently selected from the group consisting of palladium, platinum, nickel, copper, copper-chromium, rhodium, ruthenium, silver and molybdenum catalysts.

122. The process of any of claims 48-120 wherein the first and second stage catalysts are independently selected from the group consisting of palladium, platinum, and nickel catalysts.

123. The process of claim 121 or 122 wherein the first and second stage catalysts are supported on a support selected from the group consisting of alumina, carbon, titanium, silicate, silica, titania, zirconia and alumina-silica.

124. The process of any of claims 48 to 123 wherein the β-farnesene is produced by a microorganism.

125. The process of any of claims 48-124 wherein the β-farnesene incorporates carbon from a renewable carbon source.

126. The process of any of claims 48-124 wherein the β-farnesene comprises renewable carbon as determined in accordance with ASTM D6866-11.

127. The process of any of claims 48-126 wherein first stage reaction mixture comprises a diluent.

128. The process of any of claims 48-127 wherein first stage reaction product is recycled by removing it from a first, first stage reaction vessel in which the first stage reaction is being carried out and introducing it to a second, first stage reaction vessel in which the first stage reaction is being carried out.

129. The process of claim 128 wherein the first and second, first stage reaction vessels are the same reaction vessel.

130. The process of claim 128 wherein the first and second, first stage reaction vessels are different reaction vessels.

131. The process of any of claims 128 to 130 wherein the first stage recycle rate is 100% to 900% of the rate of introduction of β-farnesene to the first stage.

132. The process of any of claims 48-131 wherein the second stage reaction product is recycled by removing it from a first, second stage reaction vessel in which the second stage reaction is being carried out and introducing it to a second, second first stage reaction vessel in which the second stage reaction is being carried out.

133. The process of claim 132 wherein the first and second, second stage reaction vessels are the same reaction vessel.

134. The process of claim 132 wherein the first and second, second stage reaction vessels are different reaction vessels.

135. The process of any of claims 132 to 134 wherein the second stage recycle rate is 100% to 900% of the rate of introduction of β-farnesene to the first stage.

136. The process of any of claims 48 to 135 wherein the first stage and the second stage are independently carried out in continuous flow reactors operated adiabatically, isothermally or a combination thereof.

137. The process of claim 136 wherein the first stage is carried out in the liquid phase in one or more reaction vessels.

138. The process of claims 136 and 137 wherein the second stage is carried out in the vapor phase in one or more reaction vessels.

139. The process of any of claims 48 to 138 wherein the first stage is carried out, at least in part, in one or more continuous stirred tank reactor.
